(19) United States
(12) Patent Application Publication
(10) Pub. No.: US 2012/0164101 A1
(43) Pub. Date: Jun. 28, 2012

(54) GM-CSF AND INTERLEUKIN-21 CONJUGATES AND USES THEREOF IN THE MODULATION OF IMMUNE RESPONSE AND TREATMENT OF CANCER

(76) Inventors: Jacques Galipeau, Altanta, GA (US); Patrick Williams, Montreal (CA)

(21) Appl. No.: 13/318,066

(22) PCT Filed: Apr. 12, 2010
(86) PCT No.: PCT/CA2010/000541

§ 371 Date: Oct. 28, 2011

Related U.S. Application Data
(60) Provisional application No. 61/174,069, filed on Apr. 30, 2009.

(51) Int. Cl. A61K 38/20; C07H 21/04; C12Q 1/18; A61P 35/00; C12N 5/071; A61K 35/14; C07K 19/00; A61K 31/7088

(52) U.S. Cl. 424/85.2; 530/351; 536/23.4; 514/44 R; 435/375; 424/93.7; 424/93.71; 435/32

(57) ABSTRACT

A conjugate protein comprising a GM-CSF or fragment thereof linked to an IL-21 or fragment thereof is described. The conjugate protein has unexpected immune activating and tumoricidal properties and is useful in a variety of therapeutic applications.

### RELATED APPLICATIONS

This application claims the benefit of priority of copending U.S. provisional application No. 61/174,069 filed Apr. 30, 2009, the contents of which are incorporated herein by reference in their entirety.

### FIELD OF THE DISCLOSURE

The disclosure relates to conjugates useful in the modulation of the immune response and in treating cancer. In particular, the disclosure relates to the conjugate of GM-CSF with IL-21 and methods and uses thereof.

### BACKGROUND OF THE DISCLOSURE

Immune stimulatory cytokines can be exploited to treat human ailments including cancer. Amongst cytokines identified for such use, Granulocyte-Macrophage-Colony Stimulating Factor (GM-CSF) has been under much scrutiny since it acts directly on the adaptive immune system by enhancing antigen presentation as well as costimulation (Irvine et al. 1996). Furthermore, second generation strategies linking innate and adaptive immunity using GM-CSF delivered as a fusion cytokine (fusokine) with other immune stimulatory proteins such as Interleukin-2 (IL-2), IL-3, and IL-15 have been developed (Rafei et al. 2007). GM-CSF was first described as a growth factor for granulocyte and macrophage progenitor cells. However, GM-CSF is also an important mediator for inflammatory reactions produced by T lymphocytes, macrophages and mast cells present at sites of inflammation (Demetri and Griffin 1991). GM-CSF is a strong chemoattractant for neutrophils. It enhances microbicidal activity, phagocytotic activity and cytotoxicity of neutrophils and macrophages. An important feature of GM-CSF is that it greatly enhances the state of antigen presentation on dendritic cells, known to be crucial mediators of acquired immunity. The DNA and protein sequences of GM-CSF have been protected under PCT application WO8600639 and the derived patents.

Cellular tumor vaccines can be generated by transfecting autologous or allogeneic tumor cells with cDNAs encoding for interleukins (ILs), cytokines, interferons, and molecules accessory to immune activation (Berzofsky et al. 2004). The mechanism, by which the secretion of cytokines by tumor cells invokes an antitumor immune response, remains unclear. The reversal of “anergy,” the chemotactic, trophic, and activation effects on antigen-presenting cells, natural killer (NK) cells, macrophages, and lymphocytes at the site of an “immune” tumor vaccine are probably vital to the phenomena (Khong et al. 2002); cellular depletion studies have invoked a functional role for each of these cell types. Although the immunological underpinnings of the antitumor effect are not fully understood, phenomenological clinical studies in humans clearly demonstrate that previously immunologically silent tumors can be recognized following vaccination with tumor cells genetically engineered to express immunomodulatory proteins such as granulocyte-macrophage colony-stimulation factor (GMCSF) (Nemunaitis 2005). By comparing the antitumor effects of multiple cytokines against a mouse model of melanoma, it was found that GMCSF was the most effective cytokine in generating systemic immunity protecting against a distant tumor challenge and that IL-2 was the most effective cytokine at inducing locoregional tumor rejection (Dranoff et al. 1993). It is therefore sensible to test the combined use of GMCSF and IL-2 for cancer therapy. Because each of these cytokines has markedly distinct biological and pharmacokinetic properties, the likelihood of their contemporaneous physiological interaction with target immune effector cells is remote. The notion therefore arises that creating a fusion cytokine borne of the physical linkage of two unrelated cytokines—a “fusokine”—may possess pharmaceutical properties ascribable to each parental domain and may also acquire unheralded additive immune features. Indeed, the present inventors have previously demonstrated that a bifunctional chimeric protein borne from the fusion of GMCSF and IL-2 (hereafter GIFT-2) displayed novel and potent immunostimulatory properties that superseded those seen with either protein alone or expressed in combination (Stagg et al. 2004).

IL-21 is the most recently identified member of the common γ-chain family of cytokines, which also comprises IL-2, IL-4, IL-7, IL-9, IL-13, and IL-15 (Asao et al. 2001). IL-21's role is to promote the function of mature effector cells in the immune system. IL-21 differentiates CD4+ T cells down the Th17 pathway (Korn et al. 2007); it has been shown to activate NK cells (Roda et al. 2006) and NK cell functions like antibody-dependent cell cytotoxicity (Roda et al. 2006) and stimulate CD8+ T cells (Casey and Mescher, 2007) to mount an antitumor response (Kishida et al. 2003); furthermore, IL-21 desensitizes responder cells to the inhibitory effects of regulatory T cells (Kim-Schulze et al. 2009; Peluso et al. 2007; Li and Yee 2008), and it acts as a switch for IgG production in B cells (Konforte and Paige 2006).

The IL-21 receptor (IL-21R) is widely distributed on lympho-haematopoietic cells and IL-21 impacts a number of cell types, including CD8+ memory T cells, NK cells and subsets of CD4 memory T cells. The IL-21R has been shown to be upregulated by cells infected with HTLV. The IL-21R is expressed in leukemias and in up to 75% of follicular lymphomas, depending on the study report. The receptor is also commonly expressed in multiple myeloma, B-CLL as well as many other cancers (de Totero et al.; Blood, 2007 and Akamatsu et al.; Cancer Letters, 2007).

In certain settings, IL-6 and IL-21 are interchangeable because signaling downstream of their respective receptors is very similar, involving STAT3 and the MAP kinase pathway. Since multiple myeloma is known to produce IL-6 to promote its survival (Jernberg-Wiklund et al.; Leukemia 1990), it is therefore not a stretch to believe that IL-21 could also have pro-survival effects on multiple myeloma; indeed it has been shown that the IL-21R complex was functional in independent primary multiple myeloma samples and that IL-21 could promote cancer cell survival (Brenne et al. Blood, 2002).

Three Phase I and II clinical trials with recombinant IL-21 have been completed, providing data on the safety and efficacy in subjects with advanced melanoma, renal cell carcinoma and non-Hodgkin's B cell lymphoma. Numerous additional single-agent and combination therapy clinical trials are ongoing for a variety of human malignancies. B cell malignancies in particular warrant further clinical investigation (Andorsky and Timmerman, 2008).

The immune system is a key component of cancer genesis. Cancer suppresses responses against it by producing anti-inflammatory cytokines that prevent the activation of dendritic cells (McCormack et al., 2008). Cancer also recruits regulatory T cells (Viguier et al., 2004) and tumor associated macrophages, which in turn produce cytokines like interleukin (IL)-10 (Sica et al., 2000) and pro-angiogenic factors such as vascular endothelial growth factor (VEGF) (Crowther et al., 2001) to promote tumor growth and the suppression of T cells and dendritic cells. The ubiquitous nature of immunomodulatory mechanisms in different cancers implies there is a selection process promoting the survival and proliferation of cancer cells, meaning that this dependence is a potential vulnerability that could be widely exploited clinically. Indeed, it has been demonstrated that provided sufficient stimulation with recombinant IL-2, that it is possible to push through these barriers and induce a systemic, immune mediated anti-tumor response in patients (Rosenberg and White, 1996). Despite how chemotherapy is immunosuppressive to patients, the immune system plays a key role in limiting disease because of how chemotherapy induced cell death produces a wide array of antigens for the antigen presenting cells (APC) to process (Apetoh et al., 2007; Melief, 2008; Nowak et al., 2003).

Dendritic cells are essential for the development of all adaptive immune responses. They serve as a major platform for directing inflammation by engulfing and presenting antigens, by producing cytokines specific to events in their external environment and by providing co-stimulatory signals to T cells to modulate their reactivity towards the antigens presented. Defining different DC subsets and their interactions with T cells has been key in identifying what was necessary in order to isolate and expand a clinically useful product. While the treatments that have been developed have been successful at inducing immune responses in patients, they have not always been effective at combating established disease (Soiffer et al., 1998). More work needs to be done to find the proper conditions that will ensure that DCs possess all of the appropriate features necessary to drive an effective immune response against cancer: presenting antigen, producing cytokines and providing the proper co-stimulatory signals to T cells.

### SUMMARY OF THE DISCLOSURE

The present disclosure provides a conjugate protein where GMCSF and IL-21 have been combined and it was found that the generated molecule, GIFT-21, induces apoptosis in cancer cells expressing the IL-21R alpha chain and causes monocytes to differentiate into dendritic cells that can activate the immune response.

Accordingly, in one aspect, the present disclosure provides a conjugate protein comprising GM-CSF or a fragment thereof linked to IL-21 or a fragment thereof. In one embodiment, the GM-CSF is linked to IL-21 by a peptide linker. In another embodiment, the linker has one amino acid. In yet another embodiment, the GM-CSF lacks the last 10 carboxy terminal amino acids. In a further embodiment, the conjugate protein has the amino acid sequence shown in SEQ ID NO:2 or 4 or a homolog or analog thereof.

In another aspect, the present disclosure provides a nucleic acid molecule comprising a nucleic acid sequence encoding a conjugate protein comprising a GM-CSF or fragment thereof linked to an IL-21 or a fragment thereof. In one embodiment, the nucleic acid molecule encoding the conjugate protein has the nucleotide sequence shown in SEQ ID NO:1 or 3 or a homolog or analog thereof. In a further embodiment, the disclosure provides an expression vector comprising the nucleic acid operably linked to an expression control sequence. In yet another embodiment, the present disclosure provides a cell comprising the expression vector or progeny of said cell wherein said cell expresses the conjugate protein.

In a further aspect, the disclosure provides a method of eradicating IL-21R expressing cells such as tumor cells or activated cells of the immune system comprising administering an effective amount of a conjugate protein comprising a GM-CSF or fragment thereof linked to an IL-21 or a fragment thereof or a nucleic acid encoding the conjugate protein to an animal or cell in need thereof. In a particular embodiment, the cells express IL-21R. In a further embodiment, apoptosis in IL-21R expressing cells is induced.

In another aspect, the disclosure provides a method of activating the immune response comprising administering an effective amount of a conjugate protein comprising a GM-CSF or fragment thereof linked to an IL-21 or a fragment thereof or a nucleic acid encoding the conjugate protein to an animal or cell in need thereof.

In yet another aspect, the disclosure provides a method of treating cancer comprising administering an effective amount of a conjugate protein comprising a GM-CSF or fragment thereof linked to an IL-21 or a fragment thereof or a nucleic acid encoding the conjugate protein to an animal or cell in need thereof. The cancer can be any cancer, including without limitation, leukemia, Hodgkin's lymphoma, Non-Hodgkin's lymphoma, multiple myeloma, B-CLL as well as other IL-21R expressing cancers as well as non-hematological malignancies such as melanoma and breast cancer.

In yet a further aspect, the disclosure provides a method of activating the immune response comprising administering ex vivo-treated monocytes, dendritic cells or macrophages to an animal in need thereof, wherein the monocytes, dendritic cells or macrophages have been treated ex vivo with a conjugate protein comprising a GM-CSF or fragment thereof linked to an IL-21 or fragment thereof. In one embodiment, the method is for treating cancer or an infectious disease.

In a further aspect, the disclosure provides a pharmaceutical composition comprising an effective amount of a conjugate protein comprising a GM-CSF or fragment thereof linked to an IL-21 or fragment thereof or a nucleic acid molecule encoding the conjugate protein in admixture with a suitable diluent or carrier.

In yet a further aspect, the disclosure provides a pharmaceutical composition comprising an effective amount of ex vivo-treated monocytes, dendritic cells or macrophages in admixture with a suitable diluent or carrier, wherein the monocytes, dendritic cells or macrophages have been treated ex-vivo with a conjugate protein comprising a GM-CSF or fragment thereof linked to an IL-21 or fragment thereof.

In yet a further aspect, the disclosure provides a screening assay for determining whether or not a compound is a tumoricidal agent comprising a) incubating the compound with cells that express the IL-21R; and b) determining the effect of the compound on the induction of apoptosis via IL-21R; wherein an increase in the induction of apoptosis as compared to a control indicates that the compound is a tumoricidal agent.

In another embodiment, the disclosure provides a screening assay for determining whether or not a compound is a tumoricidal agent comprising a) incubating the compound with cells that express IL-21R in the presence of GIFT-21; and b) determining whether the compound competes with GIFT-21; wherein competition with GIFT-21 indicates that the compound is a tumoricidal agent.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The disclosure will now be described in relation to the drawings in which:

FIG. 1 shows the characterization of GIFT-21. (a) Immunoblotting performed on the CM from B16 cells retrovirally transduced to express GIFT-21; rmGMCSF and CM from B16 expressing mIL-21 were used as controls. (b) Amino acid sequence of the GIFT-21 fusokine. CM, conditioned media; GMCSF, granulocyte-macrophage colony-stimulation factor; IL-21, interleukin-21.

FIG. 2 shows the cellular biochemistry of GIFT-21. (a) STAT5 phosphorylation in RAW264.7 cells. Cells were stimulated for 20 minutes with rmGMCSF, rmIL-21, both cytokines, or with B16 GIFT-21 CM, and the cell lysates were probed for phosphorylated STAT5. Total STAT5 protein was used as a loading control. (b) STAT1/STAT3/STAT5 phosphorylation in EL-4 cells. Cells were stimulated for 20 minutes with a gradient of IL-21 and a gradient of B16 GIFT-21 CM, and the western blot of cell lysates was immunoblotted for phosphorylated STAT1 and STAT3. Total STAT1 or STAT3 protein was used as a loading control. EL-4 cells were stimulated for 20 minutes with B16 GIFT-21 CM and its controls, and the cell lysates were probed for phosphorylated STAT5. RAW264.7 cells were treated with rmGMCSF and used as a positive control for STAT5 phosphorylation in this experiment. CM, conditioned media; GMCSF, granulocyte-macrophage colony-stimulation factor; IL-21, interleukin-21.

FIG. 3 shows that GIFT-21 induces the secretion of pro-inflammatory cytokines by macrophages. (a) 5×10⁴ macrophages were treated for 24 or 48 hours with equimolar concentrations of GIFT-21 versus controls. Production of secreted IGF-1, IL-6, MCP-1, and TNF-α were measured by enzyme-linked immunosorbent assay (mean±SEM, n=3). Graphs represent the average of three independent experiments. (b) 5×10⁴ macrophages were treated with GIFT-21 with and without IL-21R blockade. GMCSF, granulocyte-macrophage colony-stimulation factor; IL-21, interleukin-21.

FIG. 4 shows that GIFT-21 induces macroscopic changes in macrophage morphology. (a) Giemsa staining of macrophages treated with GIFT-21 versus controls. Bar=50 μm. (b) Intracellular immunofluorescent staining of IL-6 (versus isotype control) in peritoneal macrophages treated with B16 GIFT-21 conditioned media (left panels) versus no treatment (right panels). Bar=10 μm. GMCSF, granulocyte-macrophage colony-stimulation factor; IL-21, interleukin-21.

FIG. 5 shows the effect of GIFT-21 on IL-21Rα expressing lymphoid subsets. Unfractionated splenocytes from normal C57BI/6 mice were treated with GIFT-21 versus controls for 24 hours; (a) apoptosis was measured by flow cytometry using annexin V and propidium iodide staining; and analysis of IL-21Rα expressing lymphocyte subsets analyzed by flow cytometry (mean±SEM, n=3); (b) CD19⁺, (c) CD3⁺CD4⁺, and (d) CD3⁺CD8⁺ cells were gated for IL-21Rα expression 24 hours following treatment with GIFT-21. Percentage of the IL-21Rα expressing fraction is indicated in top right of each flow histogram. (e) Splenocytes isolated from STAT1^{-/-} mice were treated with GIFT-21 versus controls, and the expression of the IL-21Rα was measured 24 hours later. The figures are representative of two independent experiments. GMCSF, granulocyte-macrophage colony-stimulation factor; IL-21, interleukin-21.

FIG. 6 shows that GIFT-21 elicits a robust immune response against cancer. Implantation of cytokine secreting B16 tumors in vivo. (a) B16 cells producing GIFT-21 versus all controls were implanted in WT C57BI/6 mice. B16 IL-21 and B16 GIFT-21 were robustly rejected. Data are representative of three separate experiments. (b) B16 IL-21 and B16 GIFT-21 survivors were challenged with unmodified B16, and the challenge was rejected at equal rates in both groups. (c) Only NOD-SCID mice injected with B16 GIFT-21 survived significantly longer than the controls. Data are the pooling of two separate experiments. (d) B16 GIFT-21 was rejected from STAT1^{-/-} mice. GMCSF, granulocyte-macrophage colony-stimulation factor; IL-21, interleukin-21.

FIG. 7 shows that GIFT-21 can act as an IL-21R-specific chemotherapeutic drug. (a) EL-4 express the IL-21Rα and CTLL-2 do not. (b) GIFT-21 induces the apoptosis of EL-4, but not CTLL-2 (mean±SEM, n=3). (c) Only mice treated with MSC GIFT-21 survived significantly longer than the unmodified MSC control when challenged with a 10⁶ injection of EL-4 subcutaneously. IL-21, interleukin-21; MSC, mesenchymal stromal cell; NS, not significant.

FIG. 8 shows Transmission and Scanning Electron Microscopy, light microscopy and confocal microscopy of monocytes treated for 5 days with equimolar concentrations of GIFT-21 vs controls. Monocytes treated with GIFT-21 have an enlarged endoplasmic reticulum, have long dendritic processes and bear several large LAMP-1 negative vesicles containing fluorescein dextran.

FIG. 9 shows flow cytometric characterization of conventional GMCSF+IL-4 DCs, GMCSF DCs, GMCSF+IL-21 DCs and GIFT-21 DCs.

FIG. 10 shows a: 10⁶ monocytes were differentiated using cytokines for 4 days; the cells were washed, fresh media with cytokines was added for 24 h and cytokine production was measured by ELISA for CCL2, IL-6, TNF-α and IFN-α (n=3 per group, data shown as mean+/−SEM). b: Production of mIFN-γ, as determined by ELISA, by OT-1 derived CD8⁺ T cells and OT-2 derived CD4⁺ T cells following 48 hours of co-culture with fixed, chicken ovalbumin presenting dendritic cells (n=3 per group, data shown as mean+/−SEM).

FIG. 11 shows GIFT-21 DCs migrate to tumors so they can present the antigens they sample to CCL2 recruited CD8⁺ T cells via MHCI to induce an immune response against B16 melanoma in vivo. a: C57B1/6 mice were simultaneously implanted with 5×10⁵ B16 subcutaneously and injected with RPMI or 1.5×10⁶ DCs IP. There was only a significant increase in the survival of mice treated for B16 melanoma with GIFT-21 DCs (n=10 per group, p=0.01). b: Matrigel infiltration assay. Matrigel containing 1.5×10⁶ DCs was injected subcutaneously in WT mice. Cells were recovered and stained for flow cytometry 7 days later. GIFT-21 DCs recruit significantly more CD8⁺ T cells in vivo than GMCSF+IL-4 cDCs (n=3 per group, data shown as mean+/−SEM). c: There was no significant difference in the survival of mice injected with B16 and treated with RPMI or GIFT-21 DCs in CD8−/− mice, CCR2−/− mice or in WT mice treated with β2microglobunlin deficient GIFT-21 DCs (n=5 per group). d: DAPI labeling of tumors recovered from mice injected with 1.5×10⁶ PKH26 labeled GIFT-21 DCs. Only GIFT-21 DCs infiltrated the tumors. e: Pictures of a WT C57BI/6 mouse (left) and a C57BI/6 mouse that developed vitiligo following treatment with GIFT-21 DCs for B16 melanoma (right). f: Histology of skin samples retrieved from a WT untreated C57BI/6 mouse and the mouse that developed vitiligo following treatment with GIFT-21 DCs for B16 melanoma. Tyrosinase producing melanocytes are depleted in the skin of the mouse with vitiligo.

FIG. 12 shows GIFT-21 DCs induce a CD8⁺ T cell immune response against Neu expressing D2F2 breast cancer in Balb/c mice. a: 1.5×10⁶ GIFT-21 DCs were injected IP simultaneously with 2.5×10⁵ D2F2 implanted SC in Balb/c mice (n=5 per group, data shown as mean+/−SEM). Tumor growth was significantly slowed in mice treated with GIFT-21 DCs between days 17 and 26. b: Tumor volume at day 26 was significantly smaller in the mice treated with GIFT-21 DCs than RPMI. c: Mice treated for D2F2 breast cancer had significantly more splenocytes and significantly more CD8⁺ T cells were isolated than from mice treated with RPMI (n=4 per group). d: ELISpot. CD8⁺ T cells and tumors were harvested 28 days post-implantation. CD8⁺ T cells were cultured with naïve splenocytes pulsed with the Neu 66-75 peptide. e: tumor volume was correlated with the number of Neu responsive CD8⁺ T cells in the mice that received the GIFT-21 DCs.

FIG. 13 shows a: GIFT-21 induces the upregulation of CD14 and CD80 following 5 days of treatment with GIFT-21. b: Secretion hCCL2 and hIL-6 was measured by ELISA following 5 days of incubation with GIFT-21 (n=3 per group, data shown as mean+/−SEM). c: Flow cytometry of GIFT-21 activated human monocytes incubated with fluorescein dextran. GIFT-21 induced the incorporation of fluorescein dextran.

### DETAILED DESCRIPTION OF THE DISCLOSURE

The present disclosure provides a novel fusokine, called GIFT-21, with the capacity of specifically targeting and inducing apoptosis in IL-21R-expressing cells. This novel fusokine can be used as a recombinant protein systemically or for local treatment of tumors where it can induce apoptosis of cancer cells that express IL-21R and for activating the immune response. This novel fusokine can also be used to treat monocytes, dendritic cells or macrophages ex vivo to produce dendritic cells that can activate the immune response in vivo.

### A. GM-CSF and IL-21 Conjugates

The present disclosure relates to conjugates of GM-CSF and IL-21 that can be used in various therapeutic applications as described in Section B.

Accordingly, the present disclosure provides a conjugate protein comprising a GM-CSF or a fragment thereof linked to an IL-21 or fragment thereof.

The term “IL-21” as used herein refers to interleukin 21 from any species or source and includes the full-length protein as well as fragments or portions of the protein. Mouse IL-21 has the Genbank accession number NP 068554 and human IL-21 has the Genbank accession number NP 068575. Mouse IL-21 cDNA encodes an amino acid (aa) sequence with a putative aa signal peptide that is cleaved to generate the mature protein with a molecular weight of 14.4 Da. The truncation of most of the C-terminal extension could be due to post-translational modification. IL-21 can be produced by many cells, including, without limitation, CD4 T cells and NKT cells. The term “IL-21 fragment” as used herein means a portion of the IL-21 peptide that contains at least 10%, 20%, 30%, 40%, 50%, 60%, 70%, 80%, 90%, 95%, or more of the entire length of the IL-21 polypeptide that when conjugated to GM-CSF provides for the induction of apoptosis of IL-21R-expressing cells or for activating the immune response.

The term “GM-CSF” as used herein refers to granulocyte macrophage colony stimulating factor or granulocyte macrophage colony stimulation factor from any species or source and includes the full-length protein as well as fragments or portions of the protein. Mouse GM-CSF has the Genbank accession number NM 009969 and human GM-CSF has the Genbank accession number BC108724. In one embodiment, the GM-CSF is from human or mouse. In another embodiment, the GM-CSF protein lacks the last 10 carboxy terminal amino acid sequences as compared to full length GM-CSF. The term “GM-CSF fragment” as used herein means a portion of the GM-CSF peptide that contains at least 10%, 20%, 30%, 40%, 50%, 60%, 70%, 80%, 90%, 95%, or more of the entire length of the GM-CSF polypeptide that when conjugated to IL-21 provides for the induction of apoptosis of IL-21R-expressing cells or for activating the immune response.

The term “conjugate protein” as used herein means a conjugate that comprises GM-CSF or a fragment thereof physically linked to an IL-21 or a fragment thereof, and which is able to induce apoptosis in IL-21R-expressing cells or to activate the immune response. In a specific embodiment, the conjugate is a fusion protein (or fusokine) wherein a nucleic acid sequence encoding GM-CSF or a fragment thereof is operably linked to a nucleic acid sequence encoding an IL-21 or a fragment thereof and the chimeric sequence is transfected or transduced into a host cell and produced as a recombinant fusion protein.

In an embodiment, the GM-CSF or fragment thereof and the IL-21 or fragment thereof are linked by a peptide linker. The peptide linker can be any size provided it does not interfere with the function of the conjugate protein. In one embodiment, the peptide linker is from about 1 to about 15 amino acids in length, more specifically from about 1 to about 10 amino acids, and most specifically from about 1 to about 6 amino acids. In a specific embodiment, the peptide linker forms an intercytokine bridge.

One of skill in the art can appreciate that the conjugate protein can also be formed by linking the two proteins in vitro, for example, using chemical cross-linkers. For example, the proteins may be coupled using heterobifunctional thiol-containing linkers as described in WO 90/10457, N-succinimidyl-3-(2-pyridyldithio-proprionate) or N-succinimidyl-5 thioacetate.

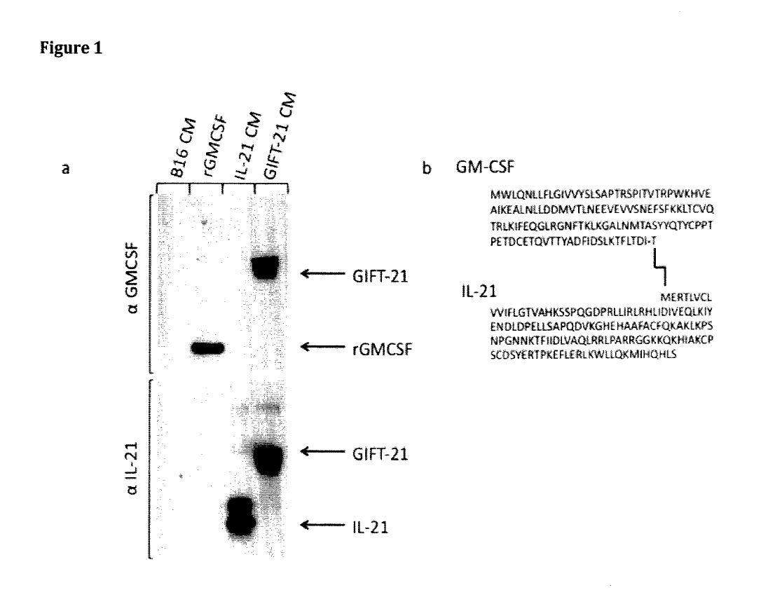

In one embodiment, the conjugate protein is murine and has the amino acid sequence shown in SEQ ID NO:2 (or FIG. 1b) or an analog or homolog thereof. This fusion protein is abbreviated GIFT-21 in the present disclosure. In another embodiment, the conjugate protein is human and has the amino acid sequence shown in SEQ ID NO:4 or an analog or homolog thereof.

The disclosure also includes nucleic acid molecules that encode the conjugate proteins described herein. The nucleic acid molecule is optionally a chimeric nucleic acid sequence that comprises a) a nucleic acid sequence encoding GM-CSF or a fragment thereof linked to b) a nucleic acid sequence encoding IL-21 or a fragment thereof.

The chimeric sequence optionally also includes a sequence encoding a peptide linker. Accordingly, the present disclosure also includes a chimeric nucleic acid sequence that comprises a) a nucleic acid sequence encoding GM-CSF or a fragment thereof linked to b) a nucleic acid sequence encoding a peptide linker linked to c) a nucleic acid sequence encoding an IL-21 or a fragment thereof.

In one embodiment, the chimeric nucleic acid sequence is murine and has the nucleotide sequence shown in SEQ ID NO:1, or a homolog or analog thereof. In another embodiment, the chimeric nucleic acid sequence is human and has the nucleotide sequence shown in SEQ ID NO:3, or a homolog or analog thereof.

The term “homolog” means those amino acid or nucleic acid sequences which have slight or inconsequential sequence variations from the sequences in SEQ ID NOs:1-4, i.e., the sequences function in substantially the same manner. The variations may be attributable to local mutations or structural modifications. Sequences having substantial homology include nucleic acid sequences having at least 65%, at least 85%, or 90-95% identity with the sequences as shown in SEQ ID NOs:1-4. Sequence identity can be calculated according to methods known in the art. Nucleic acid sequence identity is most preferably assessed by the algorithm of BLAST version 2.1 advanced search. BLAST is a series of programs that are available online at http://www.ncbi.nlm.nih.gov/BLAST. The advanced blast search (http://www.ncbi.nlm.nih.gov/blast/blast.cgi?Jform=1) is set to default parameters. (ie Matrix BLOSUM62; Gap existence cost 11; Per residue gap cost 1; Lambda ratio 0.85 default). References to BLAST searches are: Altschul, S. F., Gish, W., Miller, W., Myers, E. W. & Lipman, D. J. (1990) “Basic local alignment search tool.” J. Mol. Biol. 215:403410; Gish, W. & States, D. J. (1993) “Identification of protein coding regions by database similarity search.” Nature Genet. 3:266272; Madden, T. L., Tatusov, R. L. & Zhang, J. (1996) “Applications of network BLAST server” Meth. Enzymol. 266:131_{—}141; Altschul, S. F., Madden, T. L., Schäffer, A. A., Zhang, J., Zhang, Z., Miller, W. & Lipman, D. J. (1997) “Gapped BLAST and PSI_BLAST: a new generation of protein database search programs.” Nucleic Acids Res. 25:33893402; Zhang, J. & Madden, T. L. (1997) “PowerBLAST: A new network BLAST application for interactive or automated sequence analysis and annotation.” Genome Res. 7:649656.

The term “analog” means an amino acid or nucleic acid sequence which has been modified as compared to the sequence of SEQ ID NOs:1-4 wherein the modification does not alter the utility of the sequence (e.g. as a tumoricidal agent or immune activator) as described herein. The modified sequence or analog may have improved properties over the sequences shown in SEQ ID NOs:1-4. One example of a nucleic acid modification to prepare an analog is to replace one of the naturally occurring bases (i.e. adenine, guanine, cytosine or thymidine) of the sequence with a modified base such as xanthine, hypoxanthine, 2-aminoadenine, 6-methyl, 2-propyl and other alkyl adenines, 5-halo uracil, 5-halo cytosine, 6-aza uracil, G-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8 amino guanine, 8-thiol guanine, 8-thiolalkyl guanines, 8-hydroxyl guanine and other 8-substituted guanines, other aza and deaza uracils, thymidines, cytosines, adenines, or guanines, 5-trifluoromethyl uracil and 5-trifluoro cytosine.

Another example of a modification is to include modified phosphorous or oxygen heteroatoms in the phosphate backbone, short chain alkyl or cycloalkyl intersugar linkages or short chain heteroatomic or heterocyclic intersugar linkages in the nucleic acid molecules shown in SEQ ID NO:1 or 3. For example, the nucleic acid sequences may contain phosphorothioates, phosphotriesters, methyl phosphonates, and phosphorodithioates.

A further example of an analog of a nucleic acid molecule of the disclosure is a peptide nucleic acid (PNA) wherein the deoxyribose (or ribose) phosphate backbone in the DNA (or RNA), is replaced with a polyamide backbone which is similar to that found in peptides (P. E. Nielsen, et al Science 1991, 254, 1497). PNA analogs have been shown to be resistant to degradation by enzymes and to have extended lives in vivo and in vitro. PNAs also bind stronger to a complimentary DNA sequence due to the lack of charge repulsion between the PNA strand and the DNA strand. Other nucleic acid analogs may contain nucleotides containing polymer backbones, cyclic backbones, or acyclic backbones. For example, the nucleotides may have morpholino backbone structures (U.S. Pat. No. 5,034,506). The analogs may also contain groups such as reporter groups, a group for improving the pharmacokinetic or pharmacodynamic properties of nucleic acid sequence.

The disclosure also includes sequences that hybridize to the sequences shown in SEQ ID NO:1 or 3 or a fragment thereof and maintain the property of inducing apoptosis of IL-21R-expressing cells or activating the immune response. The term “sequence that hybridizes” means a nucleic acid sequence that can hybridize to a sequence of SEQ ID NO:1 or 3 under stringent hybridization conditions. Appropriate “stringent hybridization conditions” which promote DNA hybridization are known to those skilled in the art, or may be found in Current Protocols in Molecular Biology, John Wiley & Sons, N.Y. (1989), 6.3.1-6.3.6. The term “stringent hybridization conditions” as used herein means that conditions are selected which promote selective hybridization between two complementary nucleic acid molecules in solution. Hybridization may occur to all or a portion of a nucleic acid sequence molecule. The hybridizing portion is at least 50% the length with respect to one of the polynucleotide sequences encoding a polypeptide. In this regard, the stability of a nucleic acid duplex, or hybrids, is determined by the Tm, which in sodium containing buffers is a function of the sodium ion concentration, G/C content of labeled nucleic acid, length of nucleic acid probe (I), and temperature (Tm=81.5° C.−16.6 (Log 10 [Na+])+0.41(% (G+C)−600/l). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a greater than 95% identity, the final wash will be reduced by 5° C. Based on these considerations stringent hybridization conditions shall be defined as: hybridization at 5× sodium chloride/sodium citrate (SSC)/5×Denhardt's solution/1.0% SDS at Tm (based on the above equation)−5° C., followed by a wash of 0.2×SSC/0.1% SDS at 60° C.

It will be appreciated that analogs/homologs of the conjugate proteins described herein can also be prepared by first preparing or using an analog or homolog of GM-CSF or IL-21 or both prior to preparing the chimeric nucleic acid sequence.

The conjugate proteins described herein may be modified to contain amino acid substitutions, insertions and/or deletions that do not alter the signaling via the IL-21R properties of the protein or its property of activating the immune response. Conserved amino acid substitutions involve replacing one or more amino acids of the conjugate protein with amino acids of similar charge, size, and/or hydrophobicity characteristics. When only conserved substitutions are made the resulting analog should be functionally equivalent to the conjugate protein. Non-conserved substitutions involve replacing one or more amino acids of the conjugate protein with one or more amino acids which possess dissimilar charge, size, and/or hydrophobicity characteristics.

The conjugate proteins described herein may be modified to make it more therapeutically effective or suitable. For example, the conjugate protein or peptides of the present disclosure may be converted into pharmaceutical salts by reacting with inorganic acids including hydrochloric acid, sulphuric acid, hydrobromic acid, phosphoric acid, etc., or organic acids including formic acid, acetic acid, propionic acid, glycolic acid, lactic acid, pyruvic acid, oxalic acid, succinic acid, malic acid, tartaric acid, citric acid, benzoic acid, salicylic acid, benzenesulphonic acid, and toluenesulphonic acids.

The disclosure also includes expression vectors comprising a chimeric nucleic acid sequence comprising a) a nucleic acid sequence encoding GM-CSF or a fragment thereof linked to b) a nucleic acid sequence encoding an IL-21 or a fragment thereof. In a specific embodiment, the chimeric nucleic acid sequence includes a sequence that encodes a peptide linker as described above.

Possible expression vectors include but are not limited to cosmids, plasmids, artificial chromosomes, viral vectors or modified viruses (e.g. replication defective retroviruses, adenoviruses and adeno-associated viruses), so long as the vector is compatible with the host cell used. The expression vectors are “suitable for transformation of a host cell”, which means that the expression vectors contain a nucleic acid molecule of the disclosure and regulatory sequences selected on the basis of the host cells to be used for expression, which is operatively linked to the nucleic acid molecule. Operatively linked is intended to mean that the nucleic acid is linked to regulatory sequences in a manner which allows expression of the nucleic acid.

The disclosure therefore contemplates a recombinant expression vector of the disclosure containing a nucleic acid molecule of the disclosure, or a fragment thereof, and the necessary regulatory sequences for the transcription and translation of the inserted protein-sequence.

Suitable regulatory sequences may be derived from a variety of sources, including bacterial, fungal, viral, mammalian, or insect genes (for example, see the regulatory sequences described in Goeddel, Gene Expression Technology Methods in Enzymology 185, Academic Press, San Diego, Calif. (1990)). Selection of appropriate regulatory sequences is dependent on the host cell chosen as discussed below, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector. It will also be appreciated that the necessary regulatory sequences may be supplied by the GM-CSF or IL-21 sequences and/or their flanking regions.

The recombinant expression vectors of the disclosure may also contain a selectable marker gene which facilitates the selection of host cells transformed or transfected with a recombinant molecule of the disclosure. Examples of selectable marker genes are genes encoding a protein such as G418 and hygromycin which confer resistance to certain drugs, β-galactosidase, chloramphenicol acetyltransferase, firefly luciferase, or an immunoglobulin or portion thereof such as the Fc portion of an immunoglobulin optionally IgG. Transcription of the selectable marker gene is monitored by changes in the concentration of the selectable marker protein such as β-galactosidase, chloramphenicol acetyltransferase, or firefly luciferase. If the selectable marker gene encodes a protein conferring antibiotic resistance such as neomycin resistance transformant cells can be selected with G418. Cells that have incorporated the selectable marker gene will survive, while the other cells die. This makes it possible to visualize and assay for expression of recombinant expression vectors of the disclosure and in particular to determine the effect of a mutation on expression and phenotype. It will be appreciated that selectable markers can be introduced on a separate vector from the nucleic acid of interest.

The recombinant expression vectors may also contain genes which encode a moiety which provides increased expression of the recombinant protein; increased solubility of the recombinant protein; and aid in the purification of the target recombinant protein by acting as a ligand in affinity purification. For example, a proteolytic cleavage site may be added to the target recombinant protein to allow separation of the recombinant protein from the fusion moiety subsequent to purification of the fusion protein. Typical fusion expression vectors include pGEX (Amrad Corp., Melbourne, Australia), pMal (New England Biolabs, Beverly, Mass.) and pRIT5 (Pharmacia, Piscataway, N.J.) which fuse glutathione S-transferase (GST), maltose E binding protein, or protein A, respectively, to the recombinant protein.

Recombinant expression vectors can be introduced into host cells to produce a transformed host cell. The term “transformed host cell” is intended to include cells that are capable of being transformed or transfected with a recombinant expression vector of the disclosure. The terms “transduced”, “transformed with”, “transfected with”, “transformation” and “transfection” are intended to encompass introduction of nucleic acid (e.g. a vector or naked RNA or DNA) into a cell by one of many possible techniques known in the art. Prokaryotic cells can be transformed with nucleic acid by, for example, electroporation or calcium-chloride mediated transformation. For example, nucleic acid can be introduced into mammalian cells via conventional techniques such as calcium phosphate or calcium chloride co-precipitation, DEAE-dextran mediated transfection, lipofectin, electroporation, microinjection, RNA transfer, DNA transfer, artificial chromosomes, viral vectors and any emerging gene transfer technologies. Suitable methods for transforming and transfecting host cells can be found in Sambrook et al. (Molecular Cloning: A Laboratory Manual, 2nd Edition, Cold Spring Harbor Laboratory press (1989)), and other laboratory textbooks.

Suitable host cells include a wide variety of eukaryotic host cells and prokaryotic cells. For example, the proteins of the disclosure may be expressed in yeast cells or mammalian cells. Other suitable host cells can be found in Goeddel, Gene Expression Technology: Methods in Enzymology 185, Academic Press, San Diego, Calif. (1991). In addition, the proteins of the disclosure may be expressed in prokaryotic cells, such as Escherichia coli (Zhang et al., Science 303(5656): 371-3 (2004)).

Mammalian cells suitable for carrying out the present disclosure include, among others: B16FO cells, 293T cells, Mesenchymal Stromal Cell (MSCs), COS (e.g., ATCC No. CRL 1650 or 1651), BHK (e.g. ATCC No. CRL 6281), CHO (ATCC No. CCL 61), HeLa (e.g., ATCC No. CCL 2), 293 (ATCC No. 1573) and NS-1 cells.

The mammalian cells can also be derived from a human or animal and include stem cells (including hematopoietic stem cells), somatic cells, progenitor cells (including endothelial progenitor cells), fibroblasts, lymphocytes, and mesenchymal stem cells (MSCs) that have been genetically engineered to express the conjugate proteins described herein. Alternatively, mammalian cells can include human or animal monocytes, dendritic cells or macrophages that can be activated by the conjugate proteins of the present disclosure. The cells can be used in the therapeutic applications described in Section B.

Suitable expression vectors for directing expression in mammalian cells generally include a promoter (e.g., derived from viral material such as polyoma, Adenovirus 2, cytomegalovirus and Simian Virus 40), as well as other transcriptional and translational control sequences. Examples of mammalian expression vectors include pCDM8 (Seed, B., Nature 329:840 (1987)), pMT2PC (Kaufman et al., EMBO J. 6:187-195 (1987)) and pCMV (Clontech, California, U.S.A.).

Alternatively, the conjugate proteins of the disclosure may also be expressed in non-human transgenic animals such as, rats, rabbits, sheep and pigs (Hammer et al. Nature 315:680-683 (1985); Palmiter et al. Science 222:809-814 (1983); Brinster et al. Proc. Natl. Acad. Sci. USA 82:4438-4442 (1985); Palmiter and Brinster Cell 41:343-345 (1985) and U.S. Pat. No. 4,736,866). The disclosure also includes tissues and cells derived from such animals.

### B. Therapeutic Methods

The disclosure includes all applications of the conjugate proteins described herein, some of which are described below.

### 1. Promoting Cell Death

The present disclosure demonstrates that GIFT-21 was tumoricidal upon its addition on the murine lymphoma cell line EL4, B16 Melanoma and Neu expressing D2F2 breast cancer.

Accordingly, a conjugate protein comprising GM-CSF or a fragment thereof linked to IL-21 or a fragment thereof can be used to promote the death of a cell. In one embodiment, the present disclosure provides a method of enhancing or promoting cell death comprising administering an effective amount of a conjugate protein comprising GM-CSF or a fragment thereof linked to an IL-21 or a fragment thereof or a nucleic acid molecule encoding the conjugate protein to an animal or cell in need thereof. The disclosure includes the use of an effective amount of a conjugate protein comprising a GM-CSF or fragment thereof linked to an IL-21 or fragment thereof or a nucleic acid molecule encoding the conjugate protein to enhance or promote cell death. The disclosure also includes a use of an effective amount of a conjugate protein comprising a GM-CSF or fragment thereof linked to an IL-21 or fragment thereof or a nucleic acid molecule encoding the conjugate protein to prepare a medicament to enhance or promote cell death. The disclosure further includes a conjugate protein comprising a GM-CSF or fragment thereof linked to an IL-21 or fragment thereof or a nucleic acid molecule encoding the conjugate protein for use in enhancing or promoting cell death.

In such an embodiment, the cell may be any IL-21R expressing cell for which it is desired to promote programmed cell death. Non-limiting examples include cancer cells as well as any cell type that expresses IL-21R.

### 2. Immune Activation

Another embodiment of the present disclosure is a method for activating the immune response comprising administering an effective amount of a conjugate protein comprising a GM-CSF or fragment thereof linked to an IL-21 or fragment thereof or a nucleic acid molecule encoding the conjugate protein to an animal or cell in need thereof. The disclosure includes the use of an effective amount of a conjugate protein comprising a GM-CSF or fragment thereof linked to an IL-21 or fragment thereof or a nucleic acid molecule encoding the conjugate protein to activate the immune response. The disclosure also includes a use of an effective amount of a conjugate protein comprising a GM-CSF or fragment thereof linked to an IL-21 or fragment thereof or a nucleic acid molecule encoding the conjugate protein to prepare a medicament to activate the immune response. The disclosure further includes a conjugate protein comprising a GM-CSF or fragment thereof linked to an IL-21 or fragment thereof or a nucleic acid molecule encoding the conjugate protein for use in activating the immune response. In one embodiment, the animal or cell in need of activating the immune response has an infectious disease, a hematological cancer or a non-hematological cancer. Infectious diseases, include, without limitation, tuberculosis, Epstein Barr Virus (EBV), human immunodeficiency virus (HIV), cytomegalovirus (CMV), hepatitis C virus (HCV), herpes simplex virus (HSV), Herpes Zoster Virus (HZV), Polyoma virus, and human papilloma virus. Non-hematological cancers include, without limitation, breast cancer and melanoma. Hematological cancers include, without limitation, Hodgkin's lymphoma, leukemia, Non-Hodgkin lymphoma, multiple myeloma, and B-Chronic Lymphocytic Leukemia.

### 3. Treating Cancer

Another embodiment of the present disclosure is a method for treating cancer comprising administering an effective amount of a conjugate protein comprising a GM-CSF or fragment thereof linked to an IL-21 or fragment thereof or a nucleic acid molecule encoding the conjugate protein to an animal or cell in need thereof. The disclosure includes the use of an effective amount of a conjugate protein comprising a GM-CSF or fragment thereof linked to an IL-21 or fragment thereof or a nucleic acid molecule encoding the conjugate protein to treat cancer. The disclosure also includes a use of an effective amount of a conjugate protein comprising a GM-CSF or fragment thereof linked to an IL-21 or fragment thereof or a nucleic acid molecule encoding the conjugate protein to prepare a medicament to treat cancer. The disclosure further includes a conjugate protein comprising a GM-CSF or fragment thereof linked to an IL-21 or fragment thereof or a nucleic acid molecule encoding the conjugate protein for use in treating cancer. In one embodiment, the cancer cells are known to express IL-21R. Non-limiting examples include Hodgkin's lymphoma, leukemia, Non-Hodgkin lymphoma, multiple myeloma, and B-Chronic Lymphocytic Leukemia. In another embodiment, the cancer cells are non-hematological cancers such as breast cancer and melanoma.

The term “administering a conjugate protein” includes both the administration of the conjugate protein as well as the administration of a nucleic acid sequence encoding the conjugate protein to an animal or to a cell in vitro or in vivo. The term “administering” also includes the administration of a cell that expresses the conjugate protein.

The conjugate proteins described herein may be administered in vivo or ex vivo to a cell which is then administered. For example, cells may be transformed or transduced with the nucleic acid encoding the conjugate protein described herein and then the cells are administered in vivo. In one embodiment, the cells are mesenchymal stromal cells.

### 4. Ex-Vivo Treated Cells

The present inventors have also shown that GIFT-21 differentiates monocytes into a novel dendritic cell subtype, termed GIFT-21 DC, and that GIFT-21 DCs can be used as a cellular therapy capable of inducing a robust anti-tumor response through cell contact and secreted factors, without any prior antigen priming, following migration and sampling of the tumor. Because the phenotype of the murine cells can be replicated by applying GIFT-21 on human monocytes, these data suggest that GIFT-21 can be used to differentiate monocytes ex vivo to treat malignancy and infectious disease in humans.

Accordingly, in another embodiment, the disclosure provides a method of activating the immune response comprising administering ex vivo-treated monocytes, dendritic cells or macrophages to an animal in need thereof, wherein the monocytes, dendritic cells or macrophages have been treated ex vivo with a conjugate protein comprising a GM-CSF or fragment thereof linked to an IL-21 or fragment thereof. The disclosure also provides use of ex vivo-treated monocytes, dendritic cells or macrophages for activating the immune response in an animal in need thereof, wherein the monocytes, dendritic cells or macrophages have been treated ex vivo with a conjugate protein comprising a GM-CSF or fragment thereof linked to an IL-21 or fragment thereof. The disclosure further provides use of ex vivo-treated monocytes, dendritic cells or macrophages in the preparation of a medicament for activating the immune response in an animal in need thereof, wherein the monocytes, dendritic cells or macrophages have been treated ex vivo with a conjugate protein comprising a GM-CSF or fragment thereof linked to an IL-21 or fragment thereof. Also provided is ex vivo-treated monocytes, dendritic cells or macrophages for use in activating the immune response in an animal in need thereof, wherein the monocytes, dendritic cells or macrophages have been treated ex vivo with a conjugate protein comprising a GM-CSF or fragment thereof linked to an IL-21 or fragment thereof. In one embodiment, the method or use is for treating cancer or an infectious disease.

The term “a cell” includes a single cell as well as a plurality or population of cells. Administering to a cell includes administering in vitro (or ex vivo) as well as in vivo.

Administration of an “effective amount” of the conjugate proteins and nucleic acids or cells of the present disclosure is defined as an amount effective, at dosages and for periods of time necessary to achieve the desired result. The effective amount of the conjugate protein or nucleic acid or cells of the disclosure may vary according to factors such as the disease state, age, sex, and weight of the animal. Dosage regimens may be adjusted to provide the optimum therapeutic response. For example, several divided doses may be administered daily or the dose may be proportionally reduced as indicated by the exigencies of the therapeutic situation. The mode of administration (e.g. in vivo by injection or ex vivo in culture) will also impact the dosage regime.

The term “animal” as used herein includes all members of the animal kingdom including humans.

Once a particular conjugate protein or analog or homolog is prepared, one of skill in the art can readily determine whether or not it can promote cell death or activate the immune response. For example, determining whether a particular conjugate protein or fragments thereof can promote cell death can be assessed using known in vitro apoptotic assays, including but not limited to, calcium influx assay, induction of pro-caspase 3, chemotaxis assay, annexin V/PI costaining, and TUNEL assays and determining whether a particular conjugate protein or fragment thereof can activate the immune response can be assessed using known in vitro assays, including but not limited to, endocytosis of extra cellular products such as fluorescein dextran, antigen presentation assays or the production of pro-inflammatory cytokines such as IL-6, CCL2 or TNF-α by ELISA or flow cytometry and the analysis of cell surface markers of activation such as MHCI and the downregulation of CD11c by flow cytometry.

The term “treatment or treating” as used herein means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treating” can also mean prolonging survival as compared to expected survival if not receiving treatment.

In all of the above therapeutic applications, the conjugate protein can be administered as a protein or as a nucleic acid molecule encoding the protein. In one embodiment, as noted above, expression of the conjugate protein occurs as a result of the administration of nucleic acid encoding the conjugate protein to an organism. Thus, the conjugate protein will be produced endogenously in the organism, rather than administered in a protein form. The therapy may be done at an embryonic stage of the organism, such that the germ cells of the organism contain the conjugate protein nucleic acid, resulting in a transgenic organism, or at a later stage of development to specific somatic cells, such that only a particular tissue or portion of a tissue contains the conjugate protein nucleic acid. Techniques for nucleic acid therapy are well known in the art, as are the techniques for the creation of transgenic organisms (Carl A. Pinkert. Transgenic Animal Technology: A Laboratory Handbook. Academic Press; 1st edition (1994)).

For example, pigs and goats can be used as potential transgenic animals producing the conjugate protein. In one embodiment pigs are used in view of the fact that they possess high homology to humans in terms of MHC molecules and they are considered as a potential source of tissue and organs, in particular pancreas, heart, kidney and cornea amongst others.

It is to be understood that the administration of the conjugate protein nucleic acid in gene therapy may take several forms, all of which are included in the scope of the present disclosure. The nucleic acid encoding the conjugate protein may be administered in such a manner as to add the conjugate protein nucleic acid to the genome of the cell or the organism. For example, administering a nucleic acid encoding the conjugate protein, under the control of a promoter which results in an increase in expression of the conjugate protein, results in the incorporation of the nucleic acid into the genome of the cell or the organism, such that increased levels of the conjugate protein are made.

Construction of appropriate expression vehicles and vectors for therapeutic applications will depend on the organism to be treated and the purpose of the gene therapy. The selection of appropriate promoters and other regulatory DNA will proceed according to known principles, based on a variety of known gene therapy techniques. For example, retroviral mediated gene transfer is a very effective method for therapy, as systems utilizing packaging defective viruses allow the production of recombinants which are infectious only once, thus avoiding the introduction of wild-type virus into an organism. Alternative methodologies for therapy include non-viral transfer methods, such as calcium phosphate co-precipitation, mechanical techniques, for example microinjection, membrane fusion-mediated transfer via liposomes, as well as direct DNA uptake and receptor-mediated DNA transfer.

### C. Compositions

The disclosure includes pharmaceutical compositions containing the conjugate proteins or nucleic acids or cells described herein for use in immune activation, promoting cell death and treating cancer.

Such pharmaceutical compositions can be for intralesional, intravenous, topical, rectal, parenteral, local, inhalant or subcutaneous, intradermal, intramuscular, intrathecal, transperitoneal, oral, and intracerebral use. The composition can be in liquid, solid or semisolid form, for example pills, tablets, creams, gelatin capsules, capsules, suppositories, soft gelatin capsules, gels, membranes, tubelets, solutions or suspensions.

The pharmaceutical compositions of the disclosure can be intended for administration to humans or animals or cells or tissue in culture. Dosages to be administered depend on individual needs, on the desired effect and on the chosen route of administration.

The pharmaceutical compositions can be prepared by per se known methods for the preparation of pharmaceutically acceptable compositions which can be administered to patients, and such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 2003—20^{th} Edition) and in The United States Pharmacopeia: The National Formulary (USP 24 NF19) published in 1999).

On this basis, the pharmaceutical compositions include, albeit not exclusively, the active compound or substance in association with one or more pharmaceutically acceptable vehicles or diluents, and contained in buffered solutions with a suitable pH and iso-osmotic with the physiological fluids. The pharmaceutical compositions may additionally contain other agents such as immunoactive drugs or antibodies to enhance immune response or radio- or chemotherapeutic drugs.

In one embodiment, the pharmaceutical composition comprises an effective amount of a conjugate protein comprising a GM-CSF or fragment thereof linked to an IL-21 or fragment thereof in admixture with a pharmaceutically acceptable diluent or carrier.

In another embodiment, the pharmaceutical composition comprises an effective amount of a nucleic acid molecule encoding a conjugate protein comprising a GM-CSF or fragment thereof linked to an IL-21 or fragment thereof in admixture with a pharmaceutically acceptable diluent or carrier.

In yet another embodiment, the pharmaceutical composition comprises an effective amount of ex vivo-treated monocytes, dendritic cells or macrophages in admixture with a suitable diluent or carrier, wherein the monocytes, dendritic cells or macrophages have been treated ex-vivo with a conjugate protein comprising a GM-CSF or fragment thereof linked to an IL-21 or fragment thereof. In one embodiment, the carrier is an implantable material, such as matrigel.

### D. Screening Assay

As mentioned previously, the conjugate protein described herein exerts its effect through induction of apoptosis by the IL-21R and signaling via STAT-1. The identification of the mechanism by which the conjugate exerts its effects allows the development of screening assays that could be used to test other compounds for apoptotic or tumoricidal activity.

Accordingly, the present disclosure also provides a screening assay for determining whether or not a compound is a tumoricidal agent comprising a) incubating the compound with cells that express IL-21R; and b) determining the effect of the compound on the induction of apoptosis by the IL-21R and signaling via STAT-1 in the cells; wherein an increase of apoptosis as compared to a control indicates that the compound may be a tumoricidal agent.

Induction of apoptosis by the IL-21R and signaling via STAT-1 can be determined using techniques known in the art including apoptotic assays, including but not limited to, calcium influx assay, induction of pro-caspase 3, chemotaxis assay, annexin V/PI costaining, and TUNEL assays.

In another embodiment, the disclosure provides a screening assay for determining whether or not a compound is a tumoricidal agent comprising a) incubating the compound with cells that express IL-21R in the presence of a conjugate protein described herein; and b) determining whether the compound competes with the conjugate protein; wherein competition with the conjugate protein indicates that the compound is a tumoricidal agent.

Competition assays are known in the art. Competitive assays are widely used for different purposes such as agonist/antagonist interactions with a receptor or for concentration analysis for a drug of interest. In one example, an affinity-purified capture antibody pre-coated onto a microplate is used, to which a limited concentration of enzyme-linked analyte along with the non-labeled sample analyte are added simultaneously. Both analytes will then compete for the limited number of binding sites on the primary antibody. Substrate is added and hydrolyzed by the enzyme, thereby producing a color product that can be measured (exactly like an ELISA). The amount of labeled analyte bound is inversely proportional to the amount of unlabeled analyte presenting the sample (signal decreases as analyte concentration increases).

The test compound can be any compound which one wishes to test including, but not limited to, proteins, peptides, nucleic acids (including RNA, DNA, antisense oligonucleotide, peptide nucleic acids), carbohydrates, organic compounds, small molecules, natural products, library extracts, bodily fluids and other samples that one wishes to test for tumoricidal activity.

The cells can be any cells that either naturally express IL-21R or are transduced or transfected or otherwise engineered to express IL-21R.

Once it has been determined that a test compound or conjugate does induce apoptosis or competes with the conjugate protein described herein, it can be further tested for tumoricidal activity using techniques known in the art including the assays described herein for conjugate proteins of the disclosure.

The screening methods of the disclosure include high-throughput screening applications. For example, a high-throughput screening assay may be used which comprises any of the methods according to the disclosure wherein aliquots of cells transfected with IL-21R are exposed to a plurality of test compounds within different wells of a multi-well plate. Further, a high-throughput screening assay according to the disclosure involves aliquots of transfected cells which are exposed to a plurality of candidate conjugates in a miniaturized assay system of any kind. Another embodiment of a high-throughput screening assay could involve exposing a transduced cell population simultaneously to a plurality of test compounds.

The method of the disclosure may be “miniaturized” in an assay system through any acceptable method of miniaturization, including but not limited to multi-well plates, such as 24, 48, 96 or 384-wells per plate, micro-chips or slides. The assay may be reduced in size to be conducted on a micro-chip support, advantageously involving smaller amounts of reagent and other materials. Any miniaturization of the process which is conducive to high-throughput screening is within the scope of the disclosure.

### EXAMPLES

### Example 1

### A Fusion of GMCSF and IL-21 Initiates Hypersignaling Through the IL-21Rα Chain with Immune Activating and Tumoricidal Effects In Vivo

### Results:

### Design and Characterization of Murine GIFT-21

The fusokine (GIFT-21) was created by cloning the cDNA encoding for murine GMCSF in frame with the 5′ end of the cDNA encoding for murine IL-21. The last 30 base pairs at the 3′ end of the GMCSF cDNA were deleted to remove the stop codon, generating a cDNA encoding for a single 278 amino acid chain (FIG. 1b). Denaturing immunoblotting was performed on the conditioned media of B16 melanoma cells retrovirally transduced to express GIFT-21 (B16 GIFT-21), demonstrating that both anti-mGMCSF and anti-mIL-21 antibodies recognized the same protein at a molecular weight of ˜50 kd (FIG. 1a). It has been previously reported that murine IL-21 could be differentially glycosylated (Di Carlo et al. 2004). The IL-21 immunoblot pattern observed likely reflects this possibility.

### GIFT-21 Hyperactivates the IL-21Rα and Competitively Blocks the GMCSFR

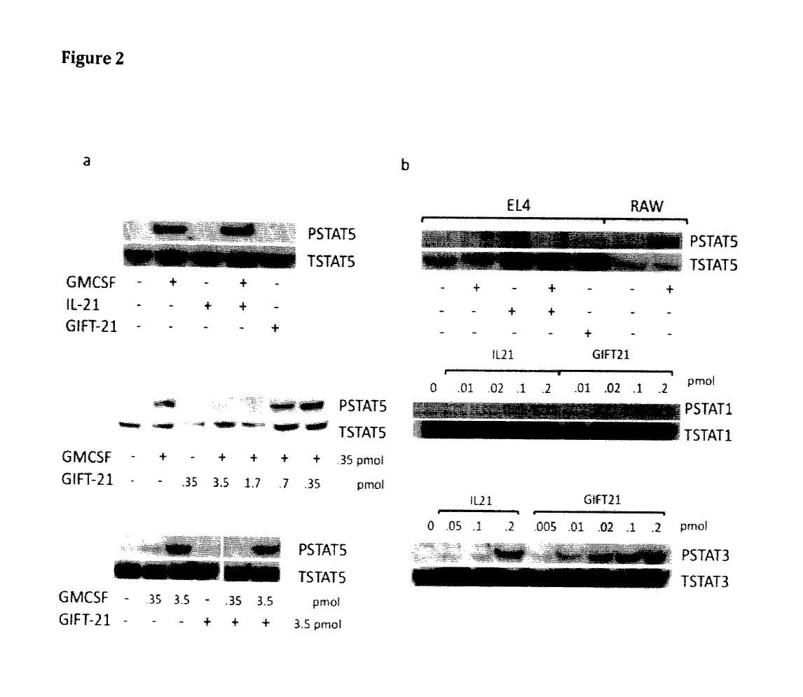

The responder murine cell lines EL-4 and RAW264.7 were utilized to analyze the cellular biochemistry of GIFT-21 on IL-21R and GMCSFR signaling independently of one another in pure populations. The murine EL-4 lymphoma cell line expresses all the components of the IL-21R (IL-21Rα and the common γ-chain) but does not express the GMCSFR. The murine myeloid RAW264.7 cell line expresses the GMCSFR, but not the IL-21Rα chain. When applying GIFT-21 conditioned media to RAW cells for 20 minutes, a lack of STAT5 activation was observed relative to the effect of GMCSF alone (FIG. 2a, top). To investigate this further, RAW cells were treated with varying ratios of GIFT-21 to rmGMCSF. GIFT-21 acted as a dominant negative to rmGMCSF by preventing rmGMCSF from inducing STAT5 phosphorylation at GIFT-21 to GMCSF molar ratios of 5:1 and 10:1 (FIG. 2a, middle and lower panels). The incubation of rmGMCSF with B16 conditioned media did not inhibit STAT5 phosphorylation in the absence of GIFT-21 (FIG. 2a, lower panel). Upon treating EL-4 cells with GIFT-21 containing conditioned media for 20 minutes, a hyperphosphorylation of IL-21Rα-dependent STAT3 was observed relative to controls, whereas STAT1 and STAT5 activation was similar to that seen with IL-21 alone (FIG. 2b). GMCSF-treated RAW cells were used as a positive control for STAT5 phosphorylation downstream of the γ-chain subunit of the IL-21R of EL-4 cells.

### GIFT-21 Rapidly Induces the Production of Pro-Inflammatory Cytokines by Macrophages

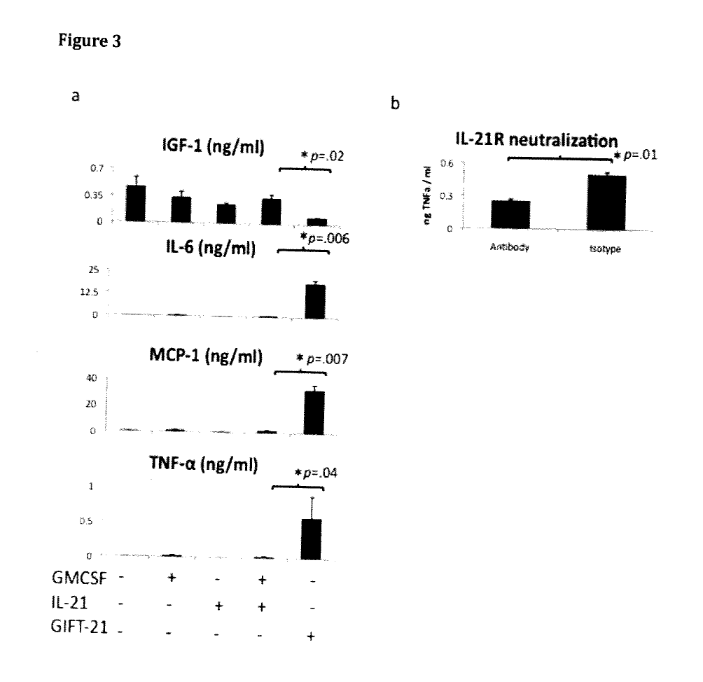

Peritoneal macrophages were harvested from the abdominal cavity of wild-type retired breeder C57BI/6 mice as previously described (Rafei et al. 2007). Enzyme-linked immunosorbent assays (ELISAs) were performed on the supernatant of peritoneal macrophages treated with cytokines for 24 and 48 hours. Following 24 hours of treatment with rmIL-21, rmGMCSF, both cytokines, or GIFT-21, IL-6, MCP-1, and TNF-α were produced in significantly larger quantities by macrophages treated with GIFT-21 than by those treated with the controls. Macrophages treated with GMCSF and IL-21 were found to produce 0.50±0.15 ng/ml IL-6, 1.4±1.1 ng/ml MCP-1, 0.014±0.025 ng/ml TNF-α, whereas GIFT-21-treated macrophages produced 19±3.2 ng/ml IL-6, 32±7.3 ng/ml MCP-1, and 0.6±0.5 ng/ml TNF-α. Macrophages treated with GIFT-21 produced significantly lower levels of IGF-1 than controls after 48 hours of incubation, 0.085±0.012 ng/ml IGF-1 as compared to 0.33±0.1 ng/ml for GMCSF and IL-21 (FIG. 3a). IL-12, IFN-γ, and TGF-β were not detected at either time points. Macrophages were treated with an anti-Fcγ blocking antibody, then with 30 μg of anti-IL-21R antibody or isotype for 2 hours, then treated with GIFT-21, and TNF-α production was measured 24 hours later. Macrophages treated with anti-IL-21R antibody produced 250 pg/ml TNF-α, significantly less TNF-α than isotype-treated macrophages, 500 pg/ml TNF-α (FIG. 3b).

### GIFT-21 Induces a Phenotypic Change in the Morphology of Macrophages

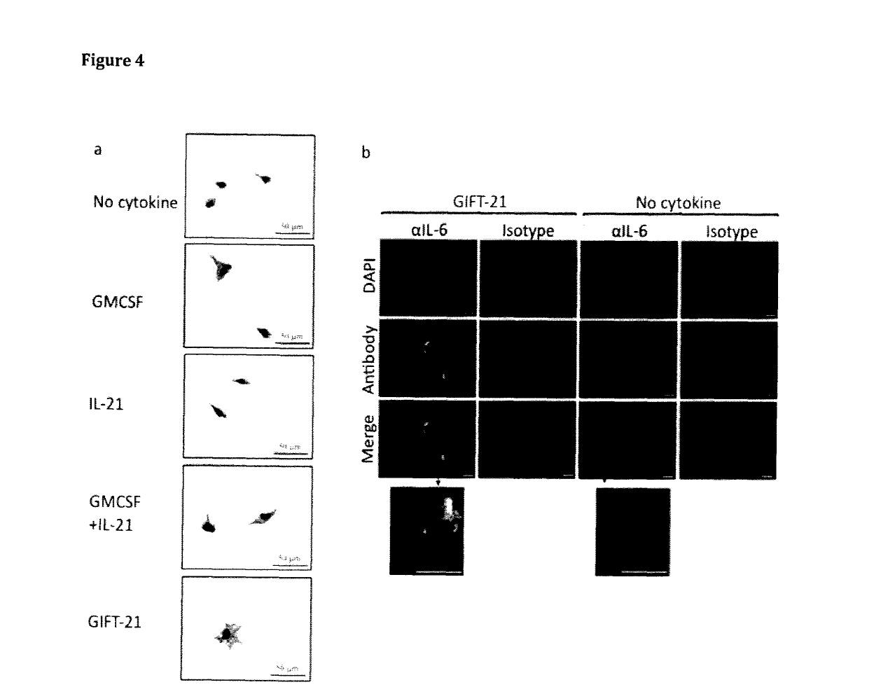

Macrophages treated with GIFT-21 were observed to adopt an altered appearance characterized by an enlarged volume, increased surface area, and the formation of long dendritic processes, vesicles, and granules (FIG. 4a). Immunofluorescent intracellular staining of GIFT-21-treated macrophages was also performed for IL-6 (FIG. 4b).

### GIFT-21 Induces Apoptosis of IL-21Rα⁺Lymphocytes in a STAT1-Dependent Manner

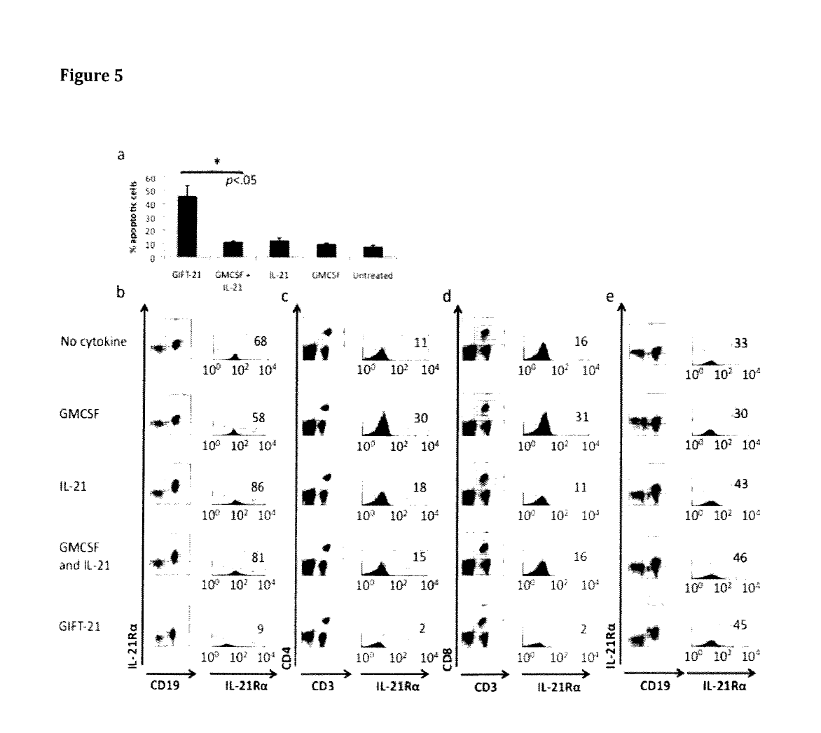

Unfractionated spleen cells were harvested from wild-type C57BI/6 mice, and 2×10⁵ splenocytes were treated in vitro with GIFT-21 and controls at equimolar concentrations (1 nmol/l) for 24 hours. GIFT-21-treated splenocytes contained significantly more annexin V positive and annexin V propidium iodide (PI) double positive cells than the controls (FIG. 5a). IL-21Rα-expressing subpopulations were gated upon in CD19+ B cells (FIG. 5b), CD3+ CD4+ (FIG. 5c), and CD3+CD8+ (FIG. 5d) T cells. A significant increase in annexin V and PI⁺ splenocytes was observed in the GIFT-21-treated group only. Compared to untreated controls, an IL-21Rα-specific depletion was observed in each lymphoid subgroup analyzed. Splenocytes were isolated from STAT1^{-/-} mice and it was found that GIFT-21 treatment had no significant effect on the number of CD19⁺/IL-21Rα⁺ B cells relative to controls (FIG. 5e).

### GIFT-21 Effect on B16 Melanoma Tumorigenicity

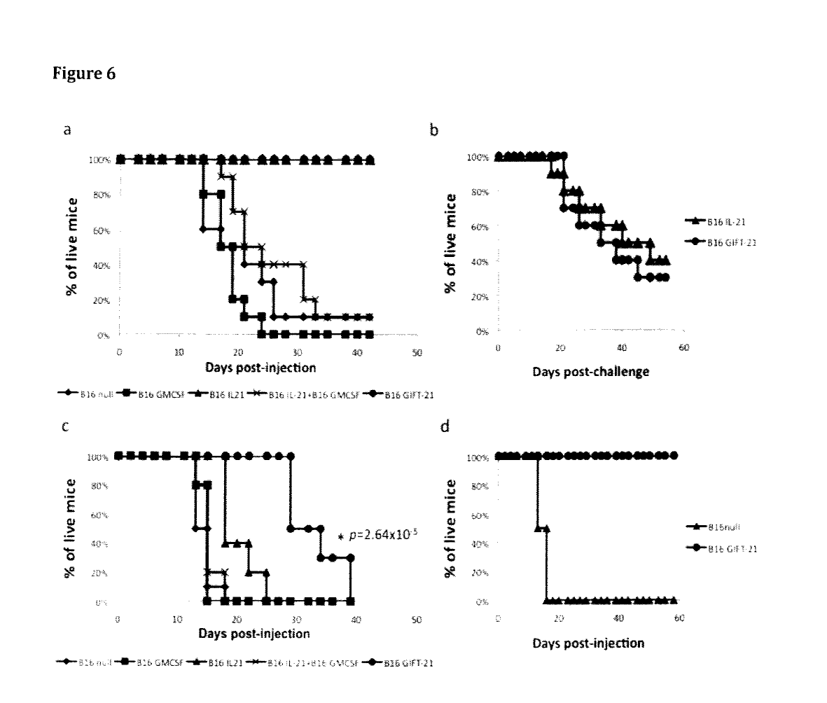

In order to evaluate GIFT-21's effectiveness as an antitumor agent in vivo, B16 melanoma cells were retrovirally transduced to express GIFT-21 (Stagg et al. 2004) and monitored survival following subcutaneous implantation in wild-type immunocompetent syngeneic C57BI/6 recipients. Five groups of mice (n=10 each) were implanted with 10⁶ gene-enhanced B16 cells: (i) untransduced B16 control; (ii) B16 expressing 5 ng/10⁶ cells/24-hour GMCSF (B16 GMCSF); (iii) B16 expressing 4 ng/10⁶ cells/24-hour IL-21 (B16 IL-21); (iv) a 1:1 mixture of B16 GMCSF and B16 IL-21; and (v) B16 expressing 2 ng/10⁶ cells/24-hour GIFT-21 (B16 GIFT-21). Both B16 GIFT-21 and B16 IL-21 groups remained tumor free for >40 days in significant contrast to all other test groups and controls (FIG. 6a). Thirty days following B16 implantation, in order to ascertain whether an adaptive response against B16 had developed, survivors of the B16 IL-21 and B16 GIFT-21 groups were then challenged with 10⁶ unmodified B16 cells subcutaneously in the contralateral flank relative to the first implantation. There was no significant difference in tumor growth between these two groups (FIG. 6b). To test the effect of GIFT-21 in mice with impaired lymphoid system, yet normal myeloid function, B16 cells were implanted subcutaneously in NOD-SCID mice and survival was monitored over time. Five groups of mice (n=10 each) were implanted with 10⁶ gene-enhanced B16 cells: (i) untransduced B16 control; (ii) B16 expressing 5 ng/10⁶ cells/24-hour GMCSF (B16 GMCSF); (iii) B16 expressing 4 ng/10⁶ cells/24-hour IL-21 (B16 IL-21); (iv) a 1:1 mixture of B16 GMCSF and B16 IL-21; and (v) B16 expressing 2 ng/10⁶ cells/24-hour GIFT-21 (B16 GIFT-21). There was no statistically significant difference in survival between the mice injected with B16 IL-21 and the rest of the controls. However, there was a significant increase in NOD-SCID mouse survival implanted with B16 GIFT-21 (logrank P=2.64×10⁻⁵) (FIG. 6c). To test whether STAT1 function is essential for rejection of B16 GIFT-21 in immunocompetent mice, 10⁶ unmodified B16 and 10⁶ B16 GIFT-21 were implanted subcutaneously STAT1^{-/-} mice (n=5) and survival was monitored over time. B16 GIFT-21 cells were rejected in 100% of STAT1^{-/-} mice (logrank P=0.002) (FIG. 6d).

### GIFT-21 Effect on IL-21Rα⁺ EL-4 Lymphoma In Vitro and In Vivo

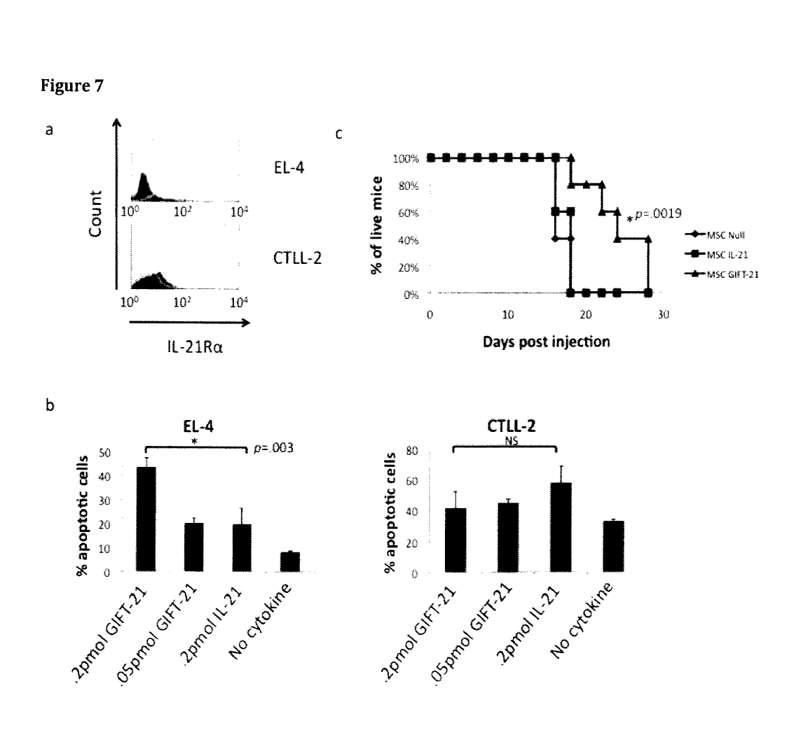

The murine EL-4 lymphoma cell line is syngeneic to the C57BI/6 strain. Cytometric analysis was performed and demonstrated that EL-4 express the IL-21Rα, and that the control CTLL-2 cells are IL-21Rα⁻ (FIG. 7a). Following in vitro treatment with GIFT-21 and rmIL-21 for 24 hours, the fraction of apoptotic cells was measured by analysis of PI/annexin V positivity by flow cytometry.

GIFT-21 induces significant dose-dependent apoptosis of IL-21Rα+EL-4 cells relative to controls and this effect is absent in IL-21Rα-CTLL-2 cells (FIG. 7b). The present inventors have previously demonstrated that mesenchymal stromal cells (MSCs) could serve as an effective vehicle for the continuous delivery of cytokines such as IL-2 and IL-12 in vivo (Stagg et al. 2004; Eliopoulos et al. 2008). Therefore, GIFT-21 engineered MSCs were used as a method to deliver the GIFT-21 fusokine systemically in mice and to determine its effect, as a plasma borne protein, on EL-4 tumor cell growth in wild-type immunocompetent C57BI/6 mice. Three test groups of C57BI/6 mice (n=5 each) were implanted intraperitoneally with gene-enhanced MSCs: (i) 12×10⁶ C57BI/6 MSCs; (ii) 12×10⁶ MSCs producing 850 pg GIFT-21/10⁶ cells/24 hours, and: (iii) 12×10⁶ MSCs producing 850 pg IL-21/10⁶ cells/24 hours. One day after MSC implantation, all mice were inoculated subcutaneously with 10⁶ EL-4 cells. Tumor volume was measured over time. A statistically significant increase in survival time was observed in mice treated with MSCs expressing GIFT-21 as compared with MSC IL-21 (P=0.0019) (FIG. 7c). There was no statistically significant difference between the unmodified MSC and MSC IL-21.

### Materials and Methods

Animals, Cell Lines, Recombinant Proteins, Antibodies, ELISA Kits, and cDNA.

Female 6- to 8-week-old C57BI/6 and C57BI/6 retired breeder mice were purchased from Harlan Laboratories (Indianapolis, Ind.), and NOD. CB17-Prkdc^{scid}/J (NOD-SCID) mice were purchased from the Jackson Laboratories (Bar Harbor, Me.). STAT1^{-/-} mice were obtained with permission from Joan Durbin. The B16F0 (B16) cell line was cultured in Dulbecco's modified Eagle's medium (DMEM) (Wisent Technologies, Rocklin, Calif.) supplemented with 10% fetal bovine serum (FBS) (Wisent Technologies) and 100 U/ml of penicillin/streptomycin (Wisent Technologies). The CTLL-2 cell line was purchased from ATCC, Manassas, Va. and was cultured in RPMI supplemented with 10% FBS, 1 mmol/l sodium pyruvate, 10% T-Stim purchased from BD Biosciences (San Diego, Calif.) and 100 U/ml of penicillin/streptomycin. The EL-4 cell line was purchased from ATCC and was cultured in RPMI (Wisent Technologies) supplemented with 10% FBS, and 100 U/ml of penicillin/streptomycin. The RAW264.7 cell line was purchased from ATCC, and it was cultured in Dulbecco's modified Eagle's medium supplemented with 10% FBS and 100 U/ml penicillin/streptomycin. Recombinant proteins (rGMCSF/mL-21), antibodies, and ELISA kits for mIL-21, mIFN-γ, mTNF-α, nnTGF-β, mIL-6, mIL-12, mJE/MCP-1, mIGF-1, mIL-21, mIL-21R, and STAT1 were purchased from R&D Systems (Minneapolis, Minn.). Anti-mouse Fcγ III/II, CD3, CD4, CD8, CD19, IL-21Rα, and isotype control antibodies for flow cytometry were purchased from BD Biosciences. Antibodies against STAT3/5 and phosphorylated STAT3/5 were purchased from Cell Signaling Technology (Danvers, Mass.). Antibodies against phosphorylated STAT1 were purchased from Abcam (Cambridge, Mass.). The mGMCSF ELISA was ordered from eBioscience (San Diego, Calif.). Apoptosis detection kits were purchased from Invitrogen (Burlington, Ontario, Canada). The murine IL-21 cDNA was purchased from Invivogen (San Diego, Calif.).

### Fusokine Design and Expression.

Mouse GMCSF cDNA was amplified by PCR, aligned in frame with the cDNA encoding IL-21, also amplified by PCR from its original vector. The GIFT-21 cDNA was cloned in a bicistronic retrovector allowing the expression of the fusokine and green fluorescent protein (Stagg et al. 2004). Infectious retroparticles were generated through transfection of 293-GP2 packaging cells (Clontech, Mountain View, Calif.) using PolyFect (Qiagen, Mississauga, Ontario, Canada). Concentrated retroparticles were used to transduce B16 and MSCs. B16 were expanded, and the supernatant was collected and concentrated using Amicon centrifugation columns (Millipore, Cambridge, Ontario, Canada). GIFT-21 expression levels were quantified using a mIL-21 ELISA kit (R&D Systems).

### GIFT-21-Mediated Biochemical Responses in Splenocytes, EL-4 and RAW264.7 Cells.

Splenocytes were collected from C57BI/6 mice. EL-4 cells were maintained in RPMI 10% FBS and RAW264.7 cells in Dulbecco's modified Eagle's medium 10% FBS. Media supplemented with cytokines was used to stimulate 2×10⁶ cells for 20 min with the different test conditions. Cell lysates were separated by 4-20% gradient SDS-PAGE (Thermo Scientific, Pittsburgh, Pa.), and western blot analysis was performed with antiphosphorylated STAT1, STAT3, and STAT5 according to manufacturer's instructions. For apoptosis assays, 2×10⁵ EL-4 cells were cultured for 24 hours and stained with PI and annexin V. Cells positive for annexin V and double positive for PI and annexin V by flow cytometric analysis were considered apoptotic. To ascertain IL-21R depletion of lymphocytes, 2×10⁵ splenocytes were cultured in a 96-well plate for 24 hours supplemented with equimolar concentrations (1 nmol/l) of GIFT-21, rmIL-21, rmGMCSF, or a combination of rmIL-21 and rmGMCSF. After incubation for 1 hour with a blocking anti-mouse Fcγ III/II antibody at 4° C., splenocytes were labeled with appropriate antibodies and analyzed by flow cytometry using a Becton Dickinson FACScan and the CellQuest software (BD Biosciences, San Jose, Calif.).

### Macrophage Collection and Phenotyping.

Peritoneal macrophages were collected by peritoneal lavage of C57BI/6 retired breeder mice. 4×10⁶ cells were then plated into 6-well flat bottom plates and left to adhere overnight in RPMI 10% FBS. The nonadherent cells were washed from the plates using sterile PBS, and the remaining adherent cells were treated with RPMI 10% FBS and equimolar concentrations of GIFT-21, recombinant mouse (rm)IL-21, rmGMCSF, or a combination of rmGMCSF and rmIL-21 for 24 and 48 hours. The cell culture medium was collected and analyzed by ELISA. In order to neutralize the IL-21R, 30 μg/ml of anti-IL-21R antibody or isotype (R&D Systems) was applied to the macrophages 2 hours prior to 24-hour stimulation with GIFT-21, and the cell culture medium was collected and analyzed by ELISA.

Peritoneal macrophages were incubated with GIFT-21 and controls in Lab-Tek II chamber slides (Nalge Nunc International, Naperville, Ill.) for 24 hours. Giemsa staining was performed by the Department of Hematology of the Jewish General Hospital. For immunofluorescence, the cells were fixed in 4% paraformaldehyde in PBS and permeabilized using 0.1% Triton X-100 in PBS. The cells were stained using an anti-mIL-6 antibody or isotype, then coupled with an Alexa 555 conjugated secondary antibody (Invitrogen Molecular Probes, Eugene, Oreg.). The slides were stained with DAPI (Invitrogen) and mounted using ImmuMount (Thermo Scientific) and visualized at room temperature using a Leica DM LB2 microscope (Leica Microsystems, Deerfield, Ill.) mounted with a DFC480 camera, using the Leica application software v3.4.0.

### Murine B16 Melanoma Modeling.

B16 cells were transduced to express GMCSF, IL-21, and GIFT-21 as previously described (Stagg et al. 2004). 10⁶ B16 cells were injected subcutaneously in syngeneic C57BI/6 mice or immunodeficient (NOD-SCID) mice, and survival was monitored over time. Mice were killed when the tumor volume reached 500 mm³ or if the tumor ulcerated. Tumor volume was measured as [(length×width²)/2]. Surviving mice were challenged with an injection of 10⁶ unmodified B16 cells subcutaneously on the contralateral flank.

### Gene-Enhanced MSCs for In Vivo Delivery of GIFT-21.

Unmodified IL-21 and GIFT-21 engineered MSCs were generated as previously described (Eliopoulos et al. 2008; Stagg et al. 2004b) and were injected intraperitoneally (12×10⁶) into naive syngeneic C57BI/6 mice. The mice were subsequently injected with 10⁶ IL-21Rα⁺EL-4 tumor cells subcutaneously 24 hours later, and tumor growth and survival was evaluated over time.

### Statistical Analysis.

P values were calculated by paired Student's t-test and logrank tests where applicable.

### Discussion

GIFT-21 was demonstrated to be more potent than IL-21 in the way that it enhances STAT3 signaling downstream of the IL-21Rα chain and, unexpectedly, it was found that this molecule acts as a dominant negative for STAT5 phosphorylation downstream of the GMCSFR. This observation suggests that GIFT-21 has unheralded immunomodulatory properties because the IL-21 and GMCSF moieties both influence the other's ability to properly bind to their respective receptor complexes.

To demonstrate GIFT-21's ability to induce an antitumor immune response, B16 melanoma was implanted into syngeneic C57BI/6 mice. IL-21 had previously been shown to be remarkably effective in eliciting an immune reaction against B16 and thus preventing the development of cancer in vivo (Ma et al. 2003; Sondergaard et al. 2007). GIFT-21 was demonstrated to also potently induce the rejection of B16. Combining B16 GMCSF with B16 IL-21 unexpectedly inhibited IL-21's pro-inflammatory properties. IL-21 has been shown to mediate part of its effects through NK cells and GMCSF is a negative regulator of NK cell activation (Faisal et al. 1990). Without wishing to be bound by any one theory, it is also possible that the combination of IL-21 and GMCSF influences local macrophages to produce cytokines that promote tumor growth instead of promoting an inflammatory response, as seen with the production of IGF-1 by peritoneal macrophages treated with GMCSF and IL-21. GMCSF has also been shown that tumors recruit myeloid-derived suppressor cells as a means of altering inflammatory reactions (Sinha et al. 2007), and it also plays a key role in the generation of myeloid-derived suppressor cells (Rössner et al. 2005). The observation that the combination of B16 GMCSF and B16 IL-21 does not induce tumor rejection stands in stark contrast to how GIFT-21 was capable of inducing robust antitumor immunity. When the surviving WT C57BI/6 mice that had been implanted with B16 GIFT-21 and B16 IL-21 were challenged, a comparable long-term antitumor memory was seen. It has previously been reported that IL-21 recruits CD8+ T cells and NK cells to mediate its effects against tumors (Ma et al. 2003), but GIFT-21 induces apoptosis of the IL-21Rα⁺ lymphoid cells it should activate. It was therefore surprising that B16 GIFT-21 would elicit tumor memory equivalent to that of B16 IL-21. Although GIFT-21's stimulatory effects were insufficient to induce the complete rejection of the tumors in NOD-SCID mice, they were sufficient to significantly increase the survival of the mice. The characterization of the macrophage response to GIFT-21 likely explains why B16 GIFT-21 elicited a partial response in NOD-SCID mice. It was therefore hypothesized that the adaptive immune system is recruited secondarily to the activation of macrophages by GIFT-21. It is believed that if the macrophages were directly killing tumor cells, the impact would have been more profound in the NOD-SCID mice because other cells would not be required to achieve the full effect seen in immunocompetent mice.

It is believed that GIFT-21 mediates its effects downstream of the IL-21R and not the GMCSFR because of how GIFT-21 not only acts as a dominant negative for GMCSFR signaling, but also because IL-21R neutralization was capable of reducing TNF-α production by primary macrophages treated with GIFT-21. It was hypothesized that STAT1 also played a key role in GIFT-21-mediated inflammation as it did in apoptosis, but B16 GIFT-21 was rejected from STAT1^{-/-} mice, indicating that GIFT-21 most likely functions by hyperphosphorylating STAT3.

The production of MCP-1, IL-6, TNF-α, IGF-1, TGF-β, IFN-γ, and IL-12 by macrophages was investigated because macrophages play a critical role in modulating the immune response. The production of these cytokines is an important part of defining classically activated macrophages involved in inflammation and alternately activated macrophages involved in wound healing (Daley et al. 2010). The production of IFN-γ and IL-12 by macrophages was also investigated because macrophages respond to LPS by producing IFN-γ and to IFN-γ by producing IL-12 in order to drive Th1 activation of CD4 T cells (Fultz et al. 1993; Hsieh et al. 1993). TNF-α and IL-6 are known to activate macrophages and lymphocytes (Andrade et al. 2005; Renauld et al. 1989) through their respective receptors and to recruit lymphocytes to areas of inflammation by inducing nearby endothelial cells to upregulate VCAM-1, permitting VLA-4-mediated migration of lymphocytes (Elices et al. 1990, lademarco et al. 1992). The production of TNF-α and IL-6 is primarily macrophage driven and the cytokines are key players in models of rheumatoid arthritis and colitis (Yamamoto et al. 2000; Alonzi et al. 1998; Firestein et al. 1990). It has also been shown that MCP-1 was an important chemokine involved in recruiting inflammatory macrophages and lymphocytes in rodent models of rheumatoid arthritis (Ogata et al. 1997; Plater-Zyberk et al. 1997). Finally, it has previously been reported that IL-21 stimulated the alternative pathway of macrophage activation, known to include the production of IGF-1, shown to be a negative regulator of TNF-α and IL-6 production by macrophages in a murine model of atherosclerosis (Sukhanov et al. 2007; Pesce et al. 2006; Wynes and Riches 2003). It is believed that GIFT-21-activated macrophages possess many features of classically activated macrophages, whereas the controls promote alternative macrophage activation. GIFT-21 provides a strong base for activating lymphoid and myeloid cells and recruiting activated lymphocytes against cancer through the production of TNF-α, IL-6, and MCP-1, and the downregulation of anti-inflammatory molecules such as IGF-1 by macrophages. Finally, the present inventors have also documented that IL-12, IFN-γ, and TGF-β were not produced under any condition.

Combining these different observations, GIFT-21 may act in a paracrine fashion through the IL-21R on the surface of myeloid cells to activate lymphocytes to mount an antitumor response with an equivalent antitumor memory as the one generated by IL-21. It is likely that lymphocytes are responding to macrophages activated by GIFT-21 and not GIFT-21 itself because of how GIFT-21 rapidly induces apoptosis of IL-21Rα+ lymphocytes. These unexpected results open up different applications for GIFT-21, either through pharmacological administration of the fusokine or through cell therapy using ex vivo—activated monocytes, dendritic cells or macrophages to treat cancer or infectious diseases that sustain themselves by suppressing the host innate immune response, such as tuberculosis (Beltan et al. 2000).

Although GIFT-21 was expected to directly amplify the functions of cytotoxic T lymphocytes and NK cells as well as enhance IL-21's modulation of antibody-dependent cell cytotoxicity, these are unlikely scenarios because GIFT-21 rapidly induces IL-21Rα lymphocytes to undergo apoptosis. It has previously been shown that IL-21 could induce apoptosis of IL-21R⁺ B-cell chronic lymphocytic leukemia, but this was only shown in vitro after the cells had been stimulated with a cocktail including CD40L or in conjunction with other drugs (Gowda et al. 2008). Furthermore, it has never been shown that IL-21 could have such effects in vivo, and the present experimental data indicate that this is unlikely as only MSC GIFT-21 could prolong the survival of mice implanted with EL-4 lymphoma. GIFT-21 carries the potential to be a receptor-specific immunotherapeutic drug. Several lymphoid malignancies, such as Hodgkin's lymphoma (Lamprecht et al. 2008), multiple myeloma (Brenne et al. 2009), and B-cell chronic lymphocytic leukemia (Lamprecht et al. 2008), often express the IL-21R. Furthermore, IL-21R signaling has been implicated in a worse prognosis in multiple myeloma (Brenne et al. 2009), making the IL-21R an excellent target for therapies utilizing GIFT-21.

Two fusokines coupling GMCSF to a common γ-chain cytokine have been previously described. The first such fusokine: GIFT-2 was characterized by its pro-inflammatory anticancer effect, mediated mainly through responsive NK cells (Stagg et al. 2004a) and altered signaling through the IL-2 receptor (Penafuerte et al. 2009). A second-generation fusokine, GIFT-15, was designed by linking GMCSF with IL-15; this paradoxically led to immune suppression due to its markedly aberrant signaling through the IL-15 receptor on lymphomyeloid cells (Rafei et al. 2007). GIFT-21 distinguishes itself from GIFT-2 and GIFT-15 by the remarkable fact that the GMCSF domain has acquired a loss-of-function phenotype, behaving as a dominant-negative inhibitor of GMCSFR signaling while also inducing hyper IL-21R signaling. The sum of these unheralded effects is a profoundly pro-inflammatory effect on myeloid cells manifested by the exuberant production of IL-6, MCP-1, and TNF-α. Despite GIFT-21's effect as an inducer of apoptosis in IL-21-Rα⁺ lymphocytes, the sum of its activating properties on myeloid cells leads to an unambiguous pro-inflammatory anticancer immune effect in vivo. The latter feature, the induction of apoptosis of IL-21Rα⁺ lymphocytes, suggested that GIFT-21 could be utilized for the selective targeting of IL-21Rα⁺ lymphoid malignancy in vivo. In conclusion, GIFT-21 represents a novel agent that co-opts IL-21R signaling in a manner useful for the enhancement of myeloid effector function as well as for targeted cancer immunotherapy.

### Example 2

### A Fusion of Granulocyte-Macrophage Colony-Stimulating Factor and Interleukin-21 Induces Monocytes to Differentiate into a Novel Dendritic Cell Population with Anti-Cancer Properties

### Results

### GIFT-21 Induced Monocyte Adherence, Growth and the Formation of Macropinocytic Vesicles

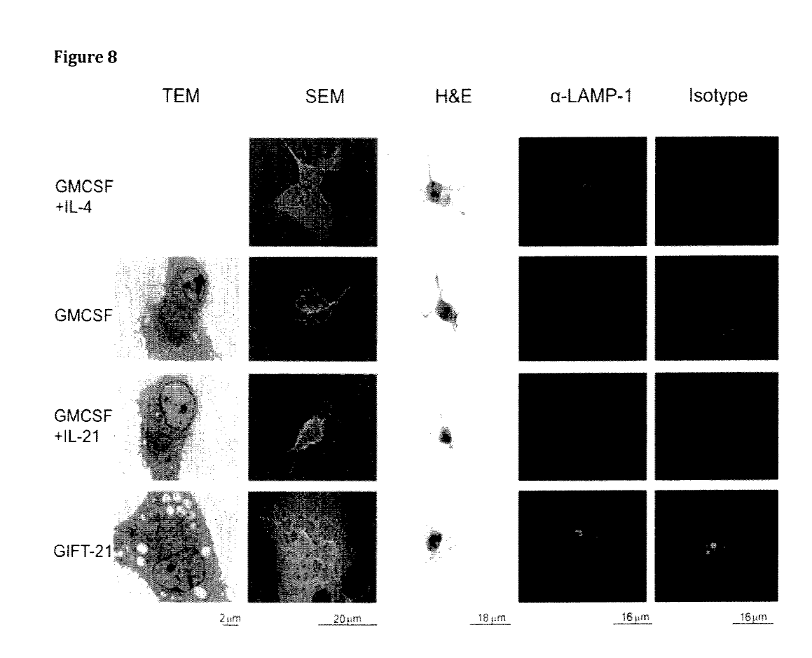

Monocytes treated with GIFT-21 adopted an altered appearance characterized by an enlarged volume, increased surface area and the formation of long dendritic processes, vesicles and granules as compared to monocytes treated with recombinant mouse (rm) GMCSF, rmGMCSF and rmIL-4 and rmGMCSF and rmIL-21. Immunofluorescent intracellular staining of GIFT-21 treated monocytes revealed that the large vesicles contained fluorescein dextran, but were negative for LAMP-1 (FIG. 8).

GIFT-21 Induced Monocytes to Differentiate into Dendritic Cells

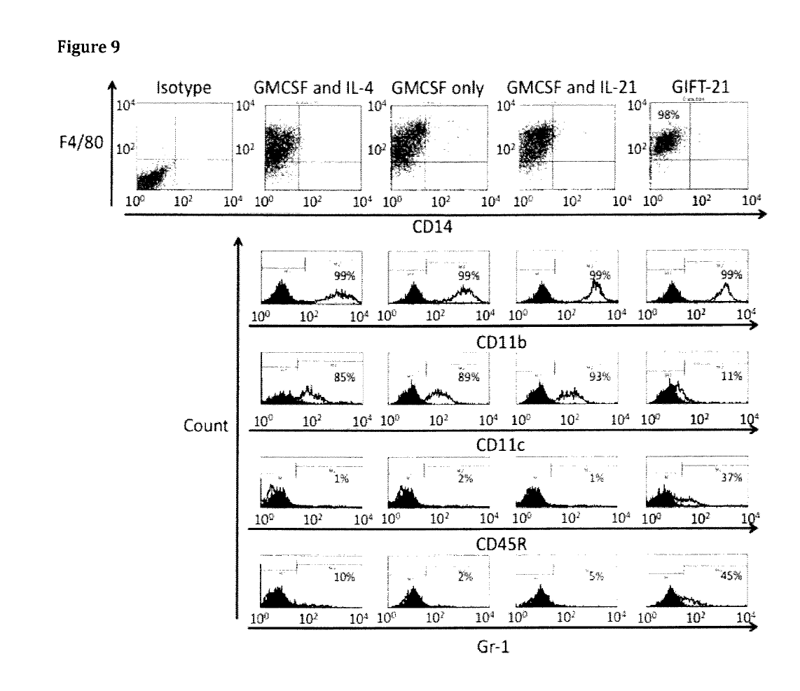

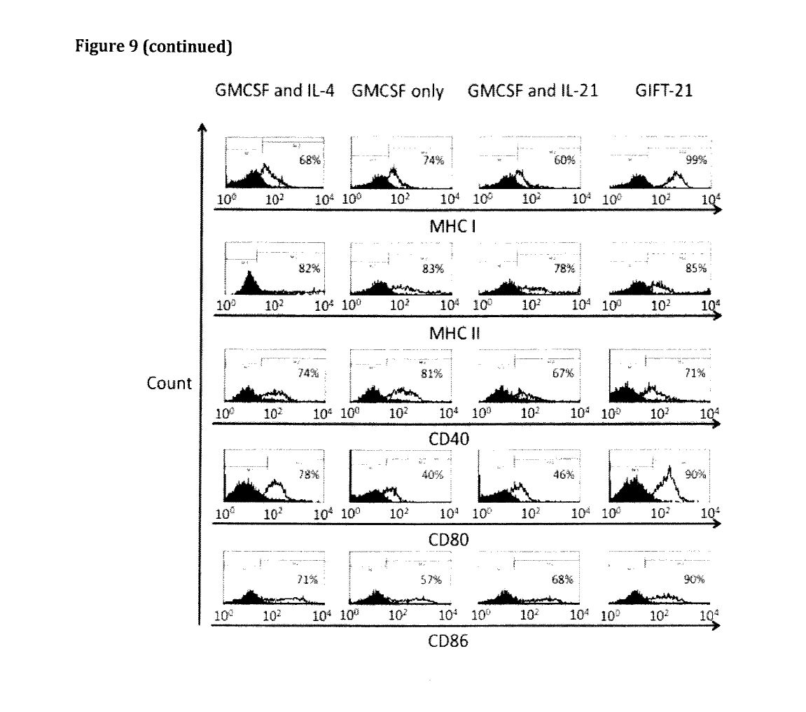

Treating monocytes with GIFT-21, rmGMCSF, the combination of rmGMCSF with rmIL-4 or rmIL-21 differentiated monocytes into dendritic cells (CD14⁻F4/80⁺). Following 5 days of culture, there was no substantial difference between monocytes treated with rmGMCSF, or the combination of rmGMCSF and rmIL-4 or rmIL-21 in terms of their surface expression of F4/80, CD14, CD11b, CD11c, Gr-1, CD45R, MHCI, MHCII, CD40, CD80, CD86. GIFT-21 dendritic cells (GIFT-21 DC) were found to have upregulated the expression of Gr-1, CD45R, MHCI and the costimulatory molecules CD80 and CD86, but downregulated CD11c and MHCII (FIG. 9).

GIFT-21 DCs Produced More Pro-Inflammatory Cytokines and Induced a Greater Production of IFN-γ by CD8⁺ T Cells in an MHCI APC Setting than cDCs

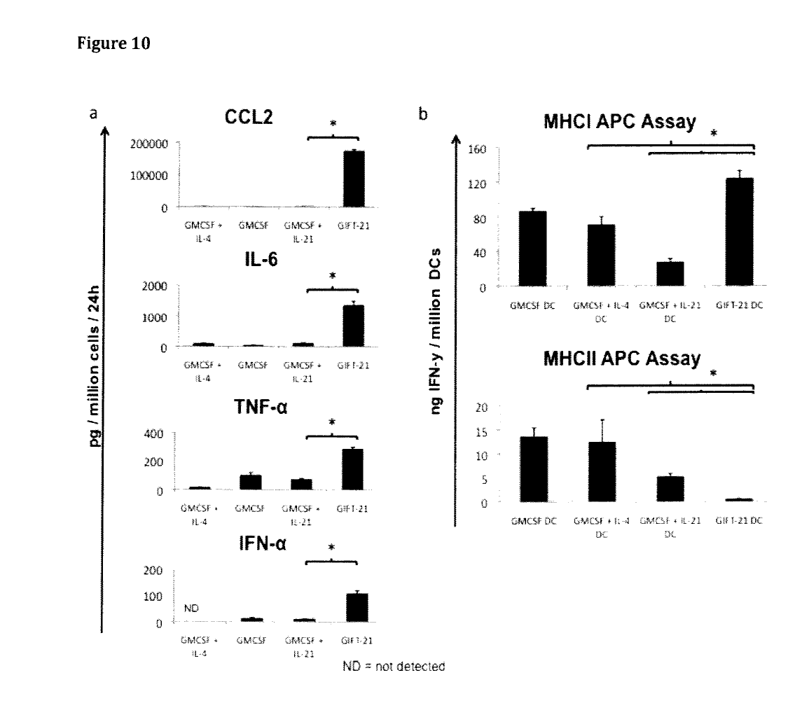

10⁶ monocytes were differentiated into DCs in 6 well plates for 4 days. The cells were washed and adherent dendritic cells were cultured with fresh cytokine containing media. Following 24 h of culture, the adherent cells were enumerated and ELISAs were performed on the supernatant of DCs. IL-6, chemokine (c-c motif) ligand (CCL)₂, tumor necrosis factor (TNF)-α and interferon (IFN)-α were produced in significantly larger quantities by GIFT-21 DCs than by the controls. 10⁶ GIFT-21 DCs produced in 24 h 300 fold more CCL2, with 170+/−7.4 ng CCL2 (p=0.0006), 15 fold more IL-6, with 1.3+/−0.21 ng IL-6 (p=0.008), 10 fold more TNF-α, with 0.28+/−0.02 ng TNF-α (p=0.03) and 110+/−0.01 ng IFN-α (p=0.01, n=3, FIG. 10a). IL-12 and IFN-γ were not detected under any condition.

The ability of DCs to present antigens through MHCI and MHCII was evaluated by measuring IFN-γ produced by T cells incubated with fixed, chicken ovalbumin presenting GIFT-21 DCs and controls for 48 h. In an MHCI restricted APC assay, CD8⁺ T cells derived from OTI mice and cultured with GIFT-21 DCs produced 130+/−9.2 ng/million DC/24 h, nearly 2 fold as much IFN-γ as CD8⁺ T cells incubated with conventional DCs (n=3, p=0.005). In an MHCII restricted APC assay, CD4⁺ T cells derived from OTII mice were cultured with GIFT-21 DCs and produced 0.60+/−0.07 ng/million DC/24 h, 20 fold less than conventional DCs (n=3, p=0.01, FIG. 10b).

### GIFT-21 DC Migrated to Tumors and Induced Rejection of B16 Melanoma in C57BI/6 Mice in a Manner Dependent Upon CD8⁺ T Cells, CCR2 and MHCI

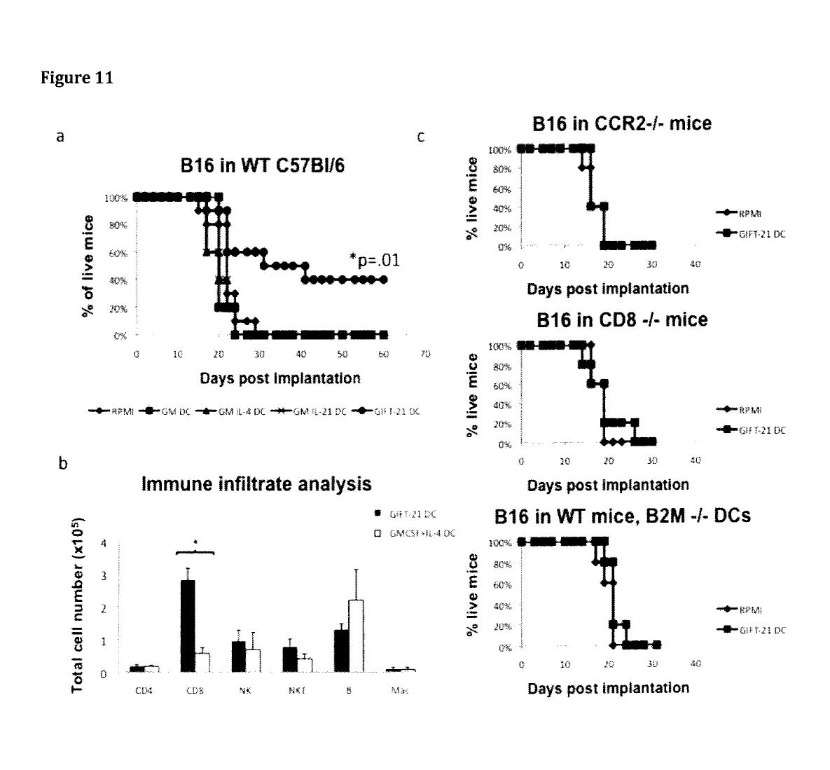

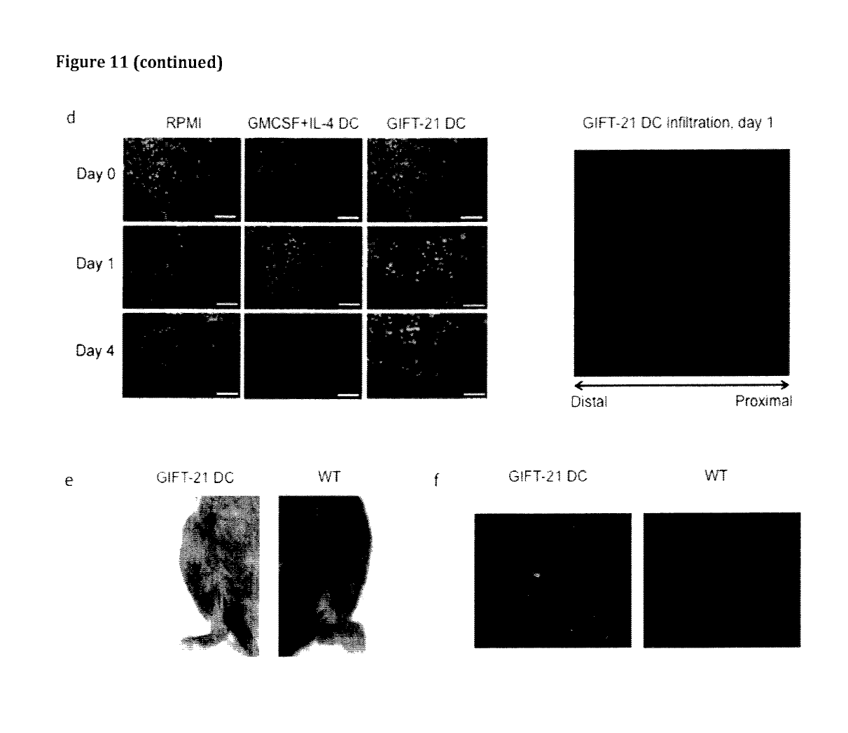

To evaluate the effectiveness of the GIFT-21 DCs at inducing an immune response against cancer in vivo, 1.5×10⁶ DCs or RPMI were injected intraperitoneally (IP) into mice also simultaneously injected with 5×10⁵ B16 subcutaneously (SC). 50% of mice treated with the GIFT-21 DCs survived to 40 days post tumor implantation, 40% of mice treated with the GIFT-21 DCs survived to 60 days post tumor implantation, while none of the controls survived past 30 days post implantation. There was no statistically significant difference between any of the controls, but the GIFT-21 DC treated mouse survival was different from that of the GMCSF IL-4 cDC treated group's at 60 days post tumor implantation (n=10 per group, p=0.01 FIG. 11a).

In order to query which cell populations were recruited by GIFT-21 DCs in vivo, 1.5×10⁶ GIFT-21 DCs or GMCSF IL-4 cDCs were injected in 400 μl matrigel SC. Flow cytometry was performed on the cells recovered from the enzymatically digested matrigel implants 7 days following implantation into C57BI/6 mice. Significantly greater numbers of CD8⁺ T cells were recovered in the implants holding GIFT-21 DCs than in the implant holdings the GMCSF IL-4 cDCs, with 2.8×10⁵+/−0.36×10⁵ in the GIFT-21 group and 0.58×10⁵+/−0.18×10⁵ in the GMCSF and IL-4 group (n=3, p=0.01, FIG. 11b).

The relationship between GIFT-21 DCs and CD8⁺ T cells was further investigated using different gene knock out models. 1.5×10⁶ GIFT-21 DCs and 5×10⁵ B16 were injected in CD8−/− and CCR2−/− mice and their survival was monitored over time. Monocytes were also isolated from β2 microglobulin deficient mice, differentiated into DCs using GIFT-21 and used to treat wild type C57BI/6 mice injected with B16 in order to see if the GIFT-21 DCs were responsible for presenting antigen to CD8⁺ T cells. No statistically significant difference was observed between the GIFT-21 DC and the RPMI groups (n=5 per group, FIG. 11c).

GIFT-21 DCs could therefore only directly induce an antigen specific response if they migrated to and sampled the tumors. B16 tumors were recovered from mice injected with PKH26 labeled DCs or RPMI and analyzed histologically and it was found that only GIFT-21 DCs migrated to the tumors; the DCs were found at the periphery proximal to the animal and did not infiltrate the core of the tumor (FIG. 11d). Remarkably, one of the five mice treated for B16 melanoma using GIFT-21 DCs began depigmenting (FIG. 11e). A skin sample was taken from this mouse and from an untreated WT C57BI/6 mouse and the depletion of tyrosinase positive melanocytes from the skin of the depigmenting mouse was confirmed (FIG. 11f). Isotype showed no staining.

GIFT-21 DCs Effectively Induce a CD13⁺ T Cell Response Against D2F2 Neu Breast Cancer

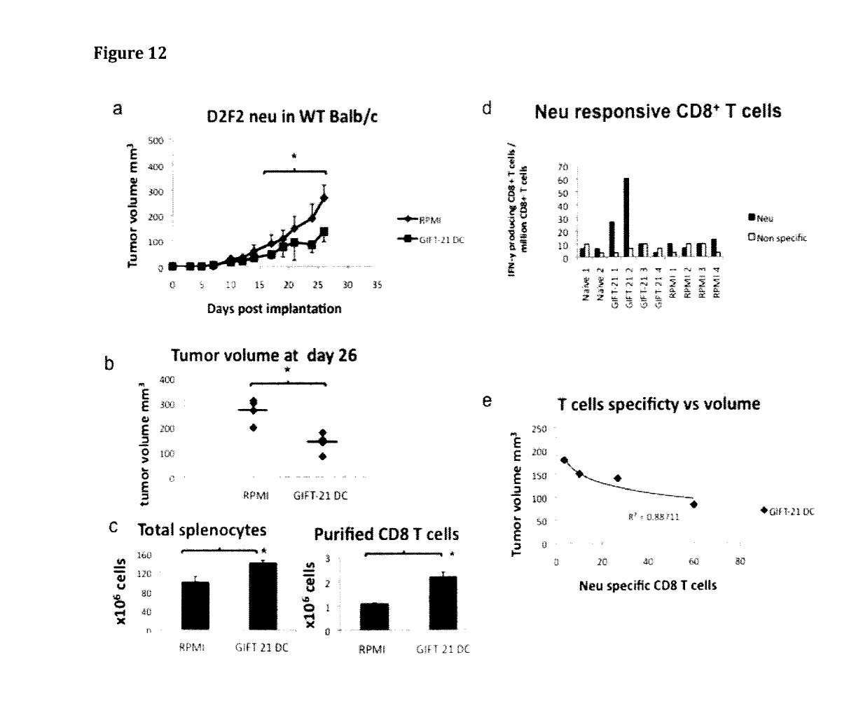

The applicability of the GIFT-21 DCs using the D2F2/neu breast cancer model in Balb/c mice was further explored, where 2.5×10⁵ D2F2 Neu breast cancer cells SC and 1.5×10⁶ GIFT-21 DCs or RPMI IP were simultaneously injected. The GIFT-21 DCs slowed the rate of tumor growth in Balb/c mice (FIG. 12a, n=4 mice per group; p<0.05 from day 17 to day 26), with the end result being that the tumors in mice treated with GIFT-21 DCs were significantly smaller than the mice injected with RPMI (n=4, p=0.0003, FIG. 12b). 28 days post implantation, the spleens of mice from each group were retrieved, CD8⁺ T cells were purified and an ELISpot analysis was performed for Neu-specific, IFN-γ producing CD8⁺ T cells. The spleens of mice treated with the GIFT-21 DCs contained significantly more splenocytes (n=4, p=0.03) and 2 fold more CD8⁺ T cells were purified from the spleens of GIFT-21 DC treated mice than RPMI (n=4, p=0.02, FIG. 12c). An ELISPOT was performed using CD8⁺ T cells co-incubated with naïve splenocytes that had been pulsed with the Neu₆₆₋₇₅ peptide and found that 2/4 mice responded to the Neu₆₆₋₇₅ peptide, whereas none of the mice treated with RPMI responded (FIG. 12d). A regression analysis on the relationship between tumor size and splenic Neu responsive CD8⁺ T cells was performed in the mice that had received GIFT-21 DCs and it was found that the two variables were correlated (R²=0.8871, FIG. 12e).

### GIFT-21 Activates Human Monocytes to Produce CCL2 and IL-6

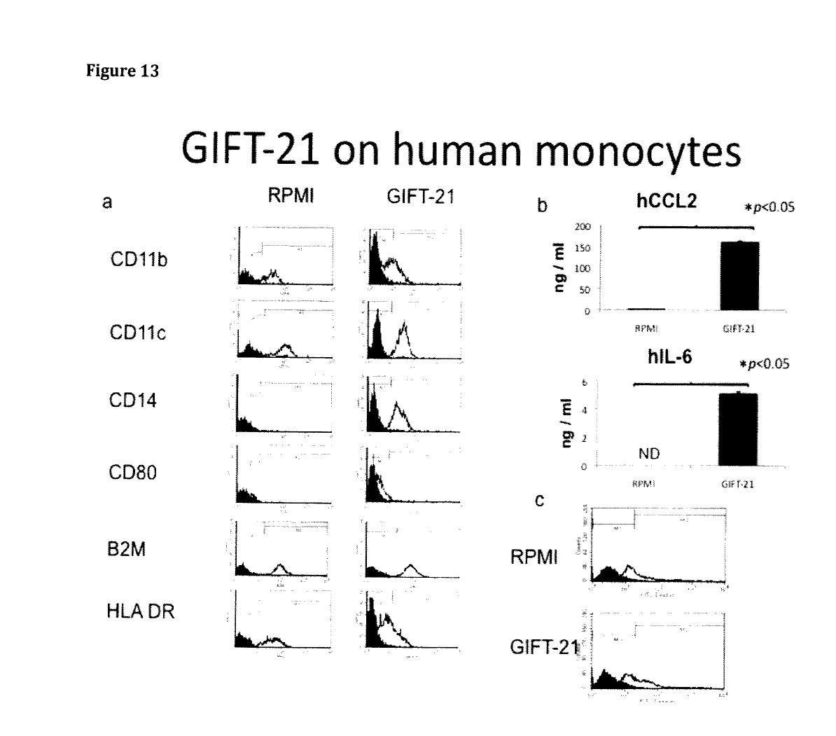

CD14⁺ monocytes were isolated from peripheral blood and treated with GIFT-21 for 5 days. The cells were characterized by flow cytometry and the supernatant was collected and analyzed for hCCL2 and hIL-6 production by ELISA. GIFT-21 induced the expression of CD14 and CD80 and the downregulation of CD11b, CD11c and HLA DR (FIG. 13a). GIFT-21 was also able to induce significantly higher levels of hCCL2 and hIL-6 expression, 160+/−2.8 ng/ml and 5.1+/−0.16 ng/ml respectively, as compared to untreated monocytes, which produced 2.3+/−0.15 ng/ml of hCCL2 and no IL-6 (FIG. 13b). Flow cytometric analysis revealed that GIFT-21 induced a greater internalization of FITC dextran than RPMI (FIG. 13c).

### Materials and Methods

### Animals and Reagents

Female 6-8 week old C57BI/6 and Balb/c mice were purchased from Harlan Laboratories. B6.129S2-Cd8a^{tm1Mak}/J (CD8−/−) mice, B6.129S4-Ccr2^{tm1lfc}/J (CCR2−/−) and B6.129P2-B2 m^{tm1Unc}/J (β2 microglobulin deficient), C57BL/6-Tg^{(TcraTcrb)1100Mjb/}J (OT-1), C57BL/6-Tg^{(TcraTcrb)425Cbn/}J (OT-2) were purchased from the Jackson Laboratories. All animal protocols were approved by the McGill University Animal Care Committee. Recombinant proteins, ELISpot and ELISA kits were purchased from R&D systems. Matrigel and antibodies for flow cytometry were purchased from BD Biosciences. All cell separations were performed using EasySep kits purchased from StemCell Technologies. Pure chicken ovalbumin (rOVA), PKH26 and dispase were purchased from Sigma-Aldrich. Fluorescein dextran, DAPI and the Alexa 555 antibody were obtained from Invitrogen. LAMP-1 antibodies were from Santa Cruz biotechnology and tyrosinase antibodies were purchased from Abcam. The rat Neu₆₆₋₇₅ (TYVPANASL, SEQ ID NO:5) peptide was obtained from the Sheldon Biotech Centre. GIFT-21 conditioned media was generated previously described (Williams et al, 2010).

### Cell Culture

The B16F0 (B16) and D2F2 Neu cell lines were cultured in DMEM supplemented with 10% FBS and 100 U/ml of Penicillin/Streptomycin.

### Dendritic Cell Differentiation and Phenotyping

Bone marrow from the femurs and tibias of mice were flushed using RPMI. The monocytic fraction was then purified using a negative selection enrichment kit. Monocytes were cultured 4 days in RPMI supplemented with 10% FBS, 100 U/ml of Penicillin/Streptomycin and 0.8 pM rmGMCSF, rmGMCSF+rmIL-4, rmGMCSF+IL-21 or GIFT-21. To phenotype the DCs, the adherent cells were washed and incubated with fresh media and cytokines for an additional 24 h. The supernatant was collected and analyzed by ELISA. The cells were gently scraped from the plate and analyzed using a BD FACScan.

### Dendritic Cell Microscopy.

For transmission electron microscopy (TEM) and scanning electron microscopy (SEM), monocyte-derived dendritic cells were cultured for 5 days and the samples were processed and analyzed by the McGill Facility for Electron Microscopy Research. H&E staining was performed by the Research Pathology Facility of the Jewish General Hospital. For confocal microscopy, the cells were treated with 25 mg/ml fluoroescein dextran for 45 minutes, then fixed and permeabilized. The cells were stained using an anti-LAMP-1 antibody or isotype then coupled with an Alexa 555 conjugated secondary antibody and stained with DAPI.

### Antigen Presentation Assay

5×10⁵ monocytes were differentiated using cytokines for 4 days in 24 well plates. The cells were then incubated with GIFT-21 or controls in RPMI supplemented with 1 mg/ml rOVA for 24 h. The cells were counted and fixed. CD4⁺ T cells and CD8⁺ T cells were isolated from the spleens of OT-2 and OT-1 mice respectively using negative selection enrichment kits. 3.5×10⁵ T cells in 500 μl RPMI supplemented with 10% FBS were applied to each sample. The supernatants were collected 48 h later and analyzed by ELISA for mIFN-γ production.

### In Vivo Cell Infiltration Assay

1.5×10⁶ DCs were mixed with 400 μl matrigel at 4° C. and injected SC in C57BI/6 mice. The implants were surgically removed 7 days later and digested in 1.6 mg/ml dispase and 200 μg/ml DNase at 37° C. into a single cell suspension. The recovered cells were washed, stained with the appropriate antibodies and analyzed by flow cytometry.

### Murine B16 Melanoma and D2F2 Breast Cancer Modeling

5×10⁵ B16 cells were injected SC in C57BI/6 mice, CD8−/− mice and CCR2−/− mice. 2.5×10⁵ D2F2 Neu cells were injected SC in Balb/c mice. 1.5×10⁵ DCs were injected IP per mouse. The importance of MHCI in vivo was studied using monocyte-derived DCs from β2 microglobulin deficient mice. Tumor volume was measured as [(length×width²)/2]. Mice were sacrificed when the tumor volume reached 500 mm³ or if the tumor ulcerated.

### Tumor Infiltration of PKH Labeled DCs

5×10⁵ B16 cells were injected SC in C57BI/6 mice. When the tumor volume reached 100 mm³, 1.5×10⁶ PKH26 labelled DCs or RPMI were injected IP. Mice were sacrificed 6 h, 24 h and 96 h later and the tumors were analyzed histologically.

### Measurement of IFN-γ Secreting CD8⁺ T Cells by ELISpot

CD8⁺ T cells were isolated from individual mice by negative selection and 1.5×10⁵ cells were incubated in 96-well mouse IFN-γ ELISPOT plates with 10⁵ naïve Balb/c mouse splenocytes that were pulsed with rat Neu₆₆₋₇₅ for 2 hours. After 48 h, the ELISPOT was developed and spots were enumerated.

### Human Monocyte Preparation

Whole blood was fractionated using Ficoll-Paque PLUS (GE Healthcare) and monocytes were extracted from buffy coat using CD14 positive selection kits. Monocytes were treated with 0.8 pM GIFT-21 for 5 days and the cells were characterized by flow cytometry; the supernatant was analyzed by ELISA for hCCL2 and hIL-6 production. Monocytes were also treated with fluorescein dextran for 45 minutes, washed and analyzed by flow cytometry.

### Statistical Analyses

P values were calculated by paired student t-test using Excel 2007 and log-rank test. Log rank testing was performed using software available at the Walter and Eliza Hall Institute website (http://bioinf.wehi.edu.au/software/russell/logrank/). Significance was defined as p<0.05 and data are reported as means+/−SEM.

### Discussion

A key hurdle in the development of tumor vaccines is the identification of antigens that can be useful at inducing an anti-tumor response (Melief, 2008). It also is not just a matter of finding any potential antigens that are specific to a given tumor, but also that can bind to the proper HLAs to promote a cytotoxic immune response (Melief, 2008). One solution to this problem has been to use irradiated tumor cells producing GMCSF to induce the recruitment and maturation of antigen presenting cells that will harvest from a broad set of antigens the tumor cells are expressing. While the vaccine was successful at inducing a measurable immune response in patients, it was not as successful at promoting cancer regression (Soiffer et al., 1998). Alternatively, other groups have used dendritic cells to prime an anti-tumor response; loading activated dendritic cells with a given antigen and using those DCs as a cell therapy, has progressed little in the clinic, either because the induced immune response was not effective (Dhodapkar et al., 2001; Jonuleit et al., 2000) or because the heterogeneity of cancer can limit the effectiveness of inducing an immune response against a single antigen (Thurner et al., 1999).

Plasmacytoid dendritic cells (pDC) are interesting in their ability to promote antigen cross-presentation by cDCs, and to recruit and activate CD8⁺ T cells and NK cells, resulting in an adaptive anti-tumor response against B16 melanoma (Liu et al., 2008). However, the same study also showed that only host derived cDCs were responsible for carrying antigens to lymph nodes for cross-presentation to CD8⁺ T cells, reinforcing how pDCs modulate the activation of other cells through the cytokines they produce instead of directly promoting an antigen specific response following cross-presentation in vivo. Most importantly, while pDCs are effective, pDCs are extremely rare (Chauhan et al., 2009) and technically challenging to isolate (Liu et al., 2008), limiting their clinical applicability.

In this study, a way around these hurdles was proposed, where it was shown that millions of GIFT-21 DCs from monocytes can be easily and quickly generated and it was shown that the GIFT-21 DCs effectively induce an adaptive anti-tumor response without prior priming. GIFT-21 DCs are unusual in how they express markers found on the surface of both cDCs and pDCs; like cDCs, the GIFT-21 DCs express CD11b and can effectively present antigen and like pDCs, they express Gr-1 and CD45R, but do not produce the same magnitude of IFN-α that have been previously associated with pDCs (Siegal et al., 1999). The cytokine secretion profile of the GIFT-21 DCs is important because CCL2 has been shown to promote the migration of macrophages (Kurihara et al., 1997) and CD8⁺ T cells (Weninger et al., 2001) and promote the survival of CD8⁺ T cells by inhibiting apoptosis (Diaz-Guerra et al., 2007). IFN-α enchances the proliferation of and decreases the apoptosis of IFN-γ producing, antigen specific and effector memory CD8⁺ T cells in cancer (Sikora et al., 2009). Furthermore, IL-6 and TNF-α can supplant CD28 to further enhance the stimulation of CD8⁺ T cells, particularly in the absence of CD4⁺ T cells (Sepulveda et al., 1999). The combination of TNF-α and IFN-α production by the GIFT-21 DCs can promote the migration of lymphocytes to areas of inflammation by inducing the expression of adhesion molecules on the surface of endothelial cells and lymphocytes; IFN-α promotes the expression of VLA-4 on the surface of T cells (Foster et al., 2004), the ligand for VCAM-1, which is induced on the surface of endothelial cells by TNF-α (Elices et al., 1990). The combined expression of all these cytokines serves to provide a pro-inflammatory environment in which the GIFT-21 DCs can effectively further stimulate CD8⁺ T cells when they are presented antigens through MHCI.

Once injected, GIFT-21 DCs have three essential properties: they migrate to the tumors, they sample their environment through macropinocytosis and they recruit CD8⁺ T cells. The presence of GIFT-21 DCs at the periphery of B16 tumors was confirmed; they were not found deeper within the tumors most likely because the cells are too large to push through the capillaries feeding the core, where the bulk of dying tissue can be found. It is likely that the reason the mice do not always reject the tumors is because the GIFT-21 DCs did not always induce an immune response against the tumor, as seen with the CD8⁺ T cells isolated from mice treated for D2F2 breast cancer by GIFT-21 DCs. Because all the mice receive the same treatment, it is unlikely due to the GIFT-21 DCs' ability to induce an immune response, but it may be because the DCs lack access to samples they could present to the immune system. Ideally, this technology would be best combined with radio- or chemotherapy, which are known to generate antigens for the DCs to present to the CD8⁺ T cells the GIFT-21 DCs attract (Apetoh et al., 2007; Chen et al., 2005; Nowak et al., 2003). Access to dying tissue is key because it can easily be sampled by the macropinosomes seen in GIFT-21 DCs. Macropinocytosis is linked with antigen cross presentation, where internalized antigens are brought to ER-like compartments containing the MHCI loading complex (Ackerman et al., 2003). Macropinosomes were identified as being large, endocytic LAMP-1⁻ vesicles (Kim et al., 2002). The GIFT-21 DCs' ability to sample fluorescein dextran in vitro and the β2 microglobulin dependence of the anti-tumor effect, indicate that unpulsed GIFT-21 DCs cross-present internalized tumor antigens through MHCI to CD8⁺ T cells following migration to the tumor, inducing an anti-tumor response against B16 melanoma and D2F2 breast cancer in vivo.

Ultimately, this results in a bimodal response to both B16 and D2F2, where either there is no difference between the controls GIFT-21 DCs or the induced immune response significantly reduced the rate of tumor growth. The anti-B16 effect observed was further emphasized when the D2F2 breast cancer model was used. The D2F2 model is syngeneic to Th2 biased Balb/c instead of Th1 biased C57BI/6, demonstrating that the GIFT-21 DCs work in different genetic and immunological backgrounds. Furthermore, the D2F2 cells express the Neu antigen, an important breast cancer subtype marker in humans, allowing the correlation of the specificity of the immune response with the global responsiveness of the mice to the treatment. While it was confirmed that two of the four GIFT-21 DC treated mice queried were reported to have mounted a response against Neu, it would be presumptuous to claim that the anti-tumor response observed in the Balb/c mice is solely due to Neu responsive CD8⁺ T cell. While it has been previously reported that under similar conditions pDCs induce cDCs to promote an anti-tumor response (Liu et al., 2008), this anti-Neu response could only have been induced by the GIFT-21 DCs because the anti-tumor effect is lost when the GIFT-21 DCs lose β2 microglobulin and thus can no longer effectively present antigens to CD8⁺ T cells. Remarkably, one of the five responding C57B1/6 mice developed a phenotype akin to vitiligo following treatment with GIFT-21 DCs for B16 melanoma. The fur of the mouse was depigmenting, suggesting that the GIFT-21 DCs trained CD8⁺ T cells cross-reacted with melanocytes in the dermis. Through a skin biopsy of the depigmenting mouse, it was found that it was devoid of tyrosinase positive melanocytes. This is significant because vitiligo is a positive predictor of survival in patients treated immunotherapeutically for melanoma (Boasberg et al., 2006; Rosenberg and White, 1996).

In another set of experiments, GIFT-21's ability to replicate the murine phenotype in human cells was investigated. Since it has previously been shown that mIL-21 could also interact with the human IL-21R (Parrish-Novak et al., 2000), human monocytes were purified from peripheral blood and treated with GIFT-21. Following treatment with GIFT-21, human monocytes did not adhere to the plates, upregulated CD14 and CD80, produced exuberant quantities of hCCL2 and hIL-6 and similarly internalized fluorescein dextran. Autologous GIFT-21 DCs may serve as a novel adjuvant in treating cancer and infectious disease.

While the disclosure has been described in connection with specific embodiments thereof, it will be understood that it is capable of further modifications and this application is intended to cover any variations, uses, or adaptations of the disclosure following, in general, the principles of the disclosure and including such departures from the present disclosures as come within known or customary practice within the art to which the disclosure pertains and as may be applied to the essential features herein before set forth, and as follows in the scope of the appended claims.

TABLE OF SEQUENCES:
MOUSE GIFT-21
DNA Sequence (SEQ ID NO: 1)
ATGTGGCTGCAGAATTTACTTTTCCTGGGCATTGTGGTCTACAGCCTCT
CAGCACCCACCCGCTCACCCATCACTGTCACCCGGCCTTGGAAGCATGT
AGAGGCCATCAAAGAAGCCCTGAACCTCCTGGATGACATGCCTGTCACG
TTGAATGAAGAGGTAGAAGTCGTCTCTAACGAGTTCTCCTTCAAGAAGC
TAACATGTGTGCAGACCCGCCTGAAGATATTCGAGCAGGGTCTACGGGG
CAATTTCACCAAACTCAAGGGCGCCTTGAACATGACAGCCAGCTACTAC
CAGACATACTGCCCCCCAACTCCGGAAACGGACTGTGAAACACAAGTTA
CCACCTATGCGGATTTCATAGACAGCCTTAAAACCTTTCTGACTGATAT
CACCATGGAGAGGACCCTTGTCTGTCTGGTAGTCATCTTCTTGGGGACA
GTGGCCCATAAATCAAGCCCCCAAGGGCCAGATCGCCTCCTGATTAGAC
TTCGTCACCTTATTGACATTGTTGAACAGCTGAAAATCTATGAAAATGA
CTTGGATCCTGAACTTCTATCAGCTCCACAAGATGTAAAGGGGCACTGT
GAGCATGCAGCTTTTGCCTGTTTTCAGAAGGCCAAACTCAAGCCATCAA
ACCCTGGAAACAATAAGACATTCATCATTGACCTCGTGGCCCAGCTCAG
GAGGAGGCTGCCTGCCAGGAGGGGAGGAAAGAAACAGAAGCACATAGCT
AAATGCCCTTCCTGTGATTCGTATGAGAAAAGGACACCCAAAGAATTCC
TAGAAAGACTAAAATGGCTCCTTCAAAAGATGATTCATCAGCATCTCTC
CTAGAACACATAG
Amino-acid Sequence (SEQ ID NO: 2)
MWLQNLLFLGIVVYSLSAPTRSPITVTRPWKHVEAIKEALNLLDDMPVT
LNEEVEVVSNEFSFKKLTCVQTRLKIFEQGLRGNFTKLKGALNMTASYY
QTYCPPTPETDCETQVTTYADFIDSLKTFLTDITMERTLVCLVVIFLGT
VAHKSSPQGPDRLLIRLRHLIDIVEQLKIYENDLDPELLSAPQDVKGHC
EHAAFACFQKAKLKPSNPGNNKTFIIDLVAQLRRRLPARRGGKKQKHIA
KCPSCDSYEKRTPKEFLERLKWLLQKMIHQHLS
Human GIFT-21
DNA Sequence (SEQ ID NO: 3)
ATGACTGCCATGTGGCTGCAGAGCCTGCTGCTCTTGGGCACTGTGGCCT
GCAGCATCTCTGCACCCGCCCGCTCGCCCAGCCCCAGCACGCAGCCCTG
GGAGCATGTGAATGCCATCCAGGAGGCCCGGCGTCTCCTGAACCTGAGT
AGAGACACTGCTGCTGAGATGAATGAAACAGTAGAAGTCATCTCAGAAA
TGTTTGACCTCCAGGAGCCGACCTGCCTACAGACCCGCCTGGAGCTGTA
CAAGCAGGGCCTGCGGGGCAGCCTCACCAAGCTCAAGGGCCCCTTGACC
ATGATGGCCAGCCACTACAAGCAGCACTGCCCTCCAACCCCGGAAACTT
CCTGTGCAACCCAGATTATCACCTTTGAAAGTTTCAAAGAGAACCTGAA
GGACTTTCTGCTTGTCATCCCCTTTGACTGCTGGGAGCCAGTCCAGGAG
TTCATGAGATCCAGTCCTGGCAACATGGAGAGGATTGTCATCTGTCTGA
TGGTCATCTTCTTGGGGACACTGGTCCACAAATCAAGCTCCCAAGGTCA
AGATCGCCACATGATTAGAATGCGTCAACTTATAGATATTGTTGATCAG
CTGAAAAATTATGTGAATGACTTGGTCCCTGAATTTCTGCCAGCTCCAG
AAGATGTAGAGACAAACTGTGAGTGGTCAGCTTTTTCCTGCTTTCAGAA
GGCCCAACTAAAGTCAGCAAATACAGGAAACAATGAAAGGATAATCAAT
GTATCAATTAAAAAGCTGAAGAGGAAACCACCTTCCACAAATGCAGGGA
GAAGACAGAAACACAGACTAACATGCCCTTCATGTGATTCTTATGAGAA
AAAACCACCCAAAGAATTCCTAGAAAGATTCAAATCACTTCTCCAAAAG
ATGATTCATCAGCATCTGTCCTCTAGAACACACGGAAGTGAAGATTCCT
GAGGATCTAACTTGCAGTTGGACAGCTAGC
Amino-acid Sequence (SEQ ID NO: 4)
MWLQSLLLLGTVACSISAPARSPSPSTQPWEHVNAIQEARRLLNLSRDT
AAEMNETVEVISEMFDLQEPTCLQTRLELYKQGLRGSLTKLKGPLTMMA
SHYKQHCPPTPETSCATQIITFESFKENLKDFLLVIPFDCWEPVQEFMR
SSPGNMERIVICLMVIFLGTLVHKSSSQGQDRHIRMRQIDIVDQLKNYV
NDLVPEFLPAPEDVETNCEWSAFSCFQKAQLKSANTGNNERIINVSIKK
LKRKPPSTNAGRRQKHRLTCPSCDSYEKKPPKEFLERFKSLLQKMIHQH
LSSRTHGSEDS

### REFERENCES

Ackerman, A. L., Kyritsis, C., Tampe, R. & Cresswell, P. Early phagosomes in dendritic cells form a cellular compartment sufficient for cross presentation of exogenous antigens. Proc Natl Acad Sci USA 100, 12889-12894 (2003).
Andorsky, D. J., and Timmerman, J. M. (2008). Interleukin-21: biology and application to cancer therapy. Expert Opin Biol Ther 8, 1295-1307.
Akamatsu N. Yamada Y, Hasegawa H, Makabe K, Asano R, Kumagai I, Murata K, Imaizumi Y, Tsukasaki K, Tsuruda K, Sugahara K, Atogami S, Yanagihara K, Kamihira S. High IL-21 receptor expression and apoptosis induction by IL-21 in follicular lymphoma. 2007. Cancer Letters. 256: 196-206
Alonzi, T, Fattori, E, Lazzaro, D, Costa, P, Probert, L, Kollias, G et al. (1998). Interleukin 6 is required for the development of collagen-induced arthritis. J Exp Med 187: 461-468.
Andrade, R M, Wessendarp, M, Portillo, J A, Yang, J Q, Gomez, F J, Durbin, J E et al. (2005). TNF receptor-associated factor 6-dependent CD40 signaling primes macrophages to acquire antimicrobial activity in response to TNF-alpha. J Immunol 175: 6014-6021.
Apetoh, L., et al. Toll-like receptor 4-dependent contribution of the immune system to anticancer chemotherapy and radiotherapy. Nat Med 13, 1050-1059 (2007).
Asao, H, Okuyama, C, Kumaki, S, Ishii, N, Tsuchiya, S, Foster, D et al. (2001). Cutting edge: the common gamma-chain is an indispensable subunit of the IL-21 receptor complex. J Immunol 167: 1-5.
Beltan, E, Horgen, L and Rastogi, N (2000). Secretion of cytokines by human macrophages upon infection by pathogenic and non-pathogenic mycobacteria. Microb Pathog 28: 313-318.
Berzofsky, J A, Terabe, M, Oh, S, Belyakov, I M, Ahlers, J D, Janik, J E et al. 2004). Progress on new vaccine strategies for the immunotherapy and prevention of cancer. J Clin Invest 113: 1515-1525.
Boasberg, P. D., et al. Enhanced survival associated with vitiligo expression during maintenance biotherapy for metastatic melanoma. J Invest Dermatol 126, 2658-2663 (2006).
Brenne A T, Baade T, Waage A, Sundan A, Borset M, Hjorth-Hansen H. Interleukin-21 is a growth and survival factor for human myeloma cells. 2002. Blood. 99: 3756-3762
Brenne, A T, Fagerli, U M, Shaughnessy, J D Jr, Vätsveen, T K, R., T B, Hella, H et al. (2009). High expression of BCL3 in human myeloma cells is associated with increased proliferation and inferior prognosis. Eur J Haematol 82: 354-363.
Casey, K A and Mescher, M F (2007). IL-21 promotes differentiation of naive CD8 T cells to a unique effector phenotype. J Immunol 178: 7640-7648.
Chauhan, D., et al. Functional interaction of plasmacytoid dendritic cells with multiple myeloma cells: a therapeutic target. Cancer Cell 16, 309-323 (2009).
Chen, Z., et al. Combined radiation therapy and dendritic cell vaccine for treating solid tumors with liver micro-metastasis. J Gene Med 7, 506-517 (2005).
Crowther, M., Brown, N. J., Bishop, E. T. & Lewis, C. E. Microenvironmental influence on macrophage regulation of angiogenesis in wounds and malignant tumors. J Leukoc Biol 70, 478-490 (2001).
Daley, J M, Brancato, S K, Thomay, A A, Reichner, J S and Albina, J E (2010). The phenotype of murine wound macrophages. J Leukoc Biol 87: 59-67.
Demetri, G. D., and Griffin, J. D. (1991). Granulocyte colony-stimulating factor and its receptor. Blood 78, 2791-2808.
de Totero D, Meazza R, Zupo S, et al. Interleukin-21 receptor (IL-21R) is up-regulated by CD40 triggering and mediates proapoptotic signals in chronic lymphocytic leukemia B cells. 2006. Blood. 107:3708-3715
Dhodapkar, M. V., Steinman, R. M., Krasovsky, J., Munz, C. & Bhardwaj, N. Antigen-specific inhibition of effector T cell function in humans after injection of immature dendritic cells. J Exp Med 193, 233-238 (2001).
Di Carlo, E, Comes, A, Orengo, A M, Rosso, O, Meazza, R, Musiani, P et al. (2004). IL-21 induces tumor rejection by specific CTL and IFN-gamma-dependent CXC chemokines in syngeneic mice. J Immunol 172: 1540-1547.
Diaz-Guerra, E., Vernal, R., del Prete, M. J., Silva, A. & Garcia-Sanz, J. A. CCL2 inhibits the apoptosis program induced by growth factor deprivation, rescuing functional T cells. J Immunol 179, 7352-7357 (2007).
Dranoff, G, Jaffee, E, Lazenby, A, Golumbek, P, Levitsky, H, Brose, K et al. (1993). Vaccination with irradiated tumor cells engineered to secrete murine granulocytemacrophage colony-stimulating factor stimulates potent, specific, and long-lasting anti-tumor immunity. Proc Natl Acad Sci USA 90: 3539-3543.
Elices, M J, Osborn, L, Takada, Y, Crouse, C, Luhowskyj, S, Hemler, M E et al. (1990). VCAM-1 on activated endothelium interacts with the leukocyte integrin VLA-4 at a site distinct from the VLA-4/fibronectin binding site. Cell 60: 577-584.
Eliopoulos, N, Francois, M, Boivin, M N, Martineau, D and Galipeau, J (2008). Neo-organoid of marrow mesenchymal stromal cells secreting interleukin-12 for breast cancer therapy. Cancer Res 68: 4810-4818.
Faisal, M, Cumberland, W, Champlin, R and Fahey, J L (1990). Effect of recombinant human granulocyte-macrophage colony-stimulating factor administration on the lymphocyte subsets of patients with refractory aplastic anemia. Blood 76: 1580-1585.
Firestein, G S, Alvaro-Gracia, J M, Maki, R and Alvaro-Garcia, J M (1990). Quantitative analysis of cytokine gene expression in rheumatoid arthritis. J Immunol 144: 3347-3353.
Foster, G. R., et al. IFN-alpha subtypes differentially affect human T cell motility. J Immunol 173, 1663-1670 (2004).
Fultz, M J, Barber, S A, Dieffenbach, C W and Vogel, S N (1993). Induction of IFNgamma in macrophages by lipopolysaccharide. Int Immunol 5: 1383-1392.
Gowda, A, Roda, J, Hussain, S R, Ramanunni, A, Joshi, T, Schmidt, S et al. (2008). IL-21 mediates apoptosis through up-regulation of the BH3 family member BIM and enhances both direct and antibody-dependent cellular cytotoxicity in primary chronic lymphocytic leukemia cells in vitro. Blood 111: 4723-4730.
Hsieh, C S, Macatonia, S E, Tripp, C S, Wolf, S F, O'Garra, A and Murphy, K M (1993). Development of TH1 CD4+ T cells through IL-12 produced by Listeria-induced macrophages. Science 260: 547-549.
Iademarco, M F, McQuillan, J J, Rosen, G D and Dean, D C (1992). Characterization of the promoter for vascular cell adhesion molecule-1 (VCAM-1). J Biol Chem 267: 16323-16329.
Irvine, K. R., Rao, J. B., Rosenberg, S. A., Restifo, N. P. (1996) Cytokine enhancement of DNA immunization leads to effective treatment of established pulmonary metastases. J. Immunol. 156, 238-245
Jernberg-Wiklund H, Pettersson M, Carlsson M, Nilsson K. Increase in IL-6 and IL-6 receptor expression in a human MM cell line, U-266, during long-term in vitro culture and the development of a possible autocrine IL-6 loop. 1992. Leukemia 6: 310-318
Jonuleit, H., Schmitt, E., Schuler, G., Knop, J. & Enk, A. H. Induction of interleukin 10-producing, nonproliferating CD4(+) T cells with regulatory properties by repetitive stimulation with allogeneic immature human dendritic cells. J Exp Med 192, 1213-1222 (2000).
Lamprecht, B, Kreher, S, Anagnostopoulos, I, Jöhrens, K, Monteleone, G, Jundt, F et al. (2008). Aberrant expression of the Th2 cytokine IL-21 in Hodgkin lymphoma cells regulates STAT3 signaling and attracts Treg cells via regulation of MIP-3alpha. Blood 112: 3339-3347.
Li, Y and Yee, C (2008). IL-21 mediated Foxp3 suppression leads to enhanced generation of antigen-specific CD8+ cytotoxic T lymphocytes. Blood 111: 229-235.
Liu, C., et al. Plasmacytoid dendritic cells induce NK cell-dependent, tumor antigen-specific T cell cross-priming and tumor regression in mice. J Clin Invest 118, 1165-1175 (2008).
Khong, H T and Restifo, N P (2002). Natural selection of tumor variants in the generation of “tumor escape” phenotypes. Nat Immunol 3: 999-1005.
Kim, S., Watarai, M., Makino, S. & Shirahata, T. Membrane sorting during swimming internalization of Brucella is required for phagosome trafficking decisions. Microb Pathog 33, 225-237 (2002).
Kim-Schulze, S, Kim, H S, Fan, Q, Kim, D W and Kaufman, H L (2009). Local IL-21 promotes the therapeutic activity of effector T cells by decreasing regulatory T cells within the tumor microenvironment. Mol Ther 17: 380-388.
Kishida, T, Asada, H, Itokawa, Y, Cui, F D, Shin-Ya, M, Gojo, S et al. (2003). Interleukin (IL)-21 and IL-15 genetic transfer synergistically augments therapeutic antitumor immunity and promotes regression of metastatic lymphoma. Mol Ther 8: 552-558.
Konforte, D and Paige, C J (2006). Identification of cellular intermediates and molecular pathways induced by IL-21 in human B cells. J Immunol 177: 8381-8392.
Korn, T, Bettelli, E, Gao, W, Awasthi, A, JaÅNger, A, Strom, T B et al. (2007). IL-21 initiates an alternative pathway to induce proinflammatory T(H)17 cells. Nature 448: 484-487.
Kurihara, T., Warr, G., Loy, J. & Bravo, R. Defects in macrophage recruitment and host defense in mice lacking the CCR2 chemokine receptor. J Exp Med 186, 1757-1762 (1997).
Ma, H L, Whitters, M J, Konz, R F, Senices, M, Young, D A, Grusby, M J et al. (2003). IL-21 activates both innate and adaptive immunity to generate potent antitumor responses that require perforin but are independent of IFN-gamma. J Immunol 171: 608-615.
McCormack, O., et al. Growth arrest-specific gene 6 expression in human breast cancer. Br J Cancer 98, 1141-1146 (2008).
Melief, C. J. Cancer immunotherapy by dendritic cells. Immunity 29, 372-383 (2008).
Nemunaitis, J (2005). Vaccines in cancer: GVAX, a GM-CSF gene vaccine. Expert Rev Vaccines 4: 259-274.
Nowak, A. K., Robinson, B. W. & Lake, R. A. Synergy between chemotherapy and immunotherapy in the treatment of established murine solid tumors. Cancer Res 63, 4490-4496 (2003).
Ogata, H, Takeya, M, Yoshimura, T, Takagi, K and Takahashi, K (1997). The role of monocyte chemoattractant protein-1 (MCP-1) in the pathogenesis of collagen-induced arthritis in rats. J Pathol 182: 106-114.
Parrish-Novak, J., et al. Interleukin 21 and its receptor are involved in NK cell expansion and regulation of lymphocyte function. Nature 408, 57-63 (2000).
Peluso, I, Fantini, M C, Fina, D, Caruso, R, Boirivant, M, MacDonald, T T at al. (2007). IL-21 counteracts the regulatory T cell-mediated suppression of human CD4+ T lymphocytes. J Immunol 178: 732-739.
Penafuerte, C, Bautista-Lopez, N, Mohamed-Rachid, B, Routy, J P and Galipeau, J (2009). The human ortholog of granulocyte macrophage colony-stimulating factor and interleukin-2 fusion protein induces potent ex vivo natural killer cell activation and maturation. Cancer Res 69: 9020-9028.
Pesce, J, Kaviratne, M, Ramalingam, T R, Thompson, R W, Urban, J F Jr, Cheever, A W at al. (2006). The IL-21 receptor augments Th2 effector function and alternative macrophage activation. J Clin Invest 116: 2044-2055.
Plater-Zyberk, C, Hoogewerf, A J, Proudfoot, A E, Power, C A and Wells, T N (1997). Effect of a CC chemokine receptor antagonist on collagen induced arthritis in DBA/1 mice. Immunol Lett 57: 117-120.
Rafei, M, Wu, J H, Annabi, B, Lejeune, L, Francois, M and Galipeau, J (2007). A GMCSF and IL-15 fusokine leads to paradoxical immunosuppression in vivo via asymmetrical JAK/STAT signaling through the IL-15 receptor complex. Blood 109: 2234-2242.
Renauld, J C, Vink, A and Van Snick, J (1989). Accessory signals in murine cytolytic T cell responses. Dual requirement for IL-1 and IL-6. J Immunol 143:1894-1898.
Roda, J M, Parihar, R, Lehman, A, Mani, A, Tridandapani, S and Carson, W E 3rd (2006). Interleukin-21 enhances NK cell activation in response to antibody-coated targets. J Immunol 177: 120-129.
Rosenberg, S. A. & White, D. E. Vitiligo in patients with melanoma: normal tissue antigens can be targets for cancer immunotherapy. J Immunother Emphasis Tumor Immunol 19, 81-84 (1996).
Rössner, S, Voigtländer, C, Wiethe, C, Hänig, J, Seifarth, C and Lutz, M B (2005). Myeloid dendritic cell precursors generated from bone marrow suppress T cell responses via cell contact and nitric oxide production in vitro. Eur J Immunol 35: 3533-3544.
Sepulveda, H., Cerwenka, A., Morgan, T. & Dutton, R. W. CD28, IL-2-independent costimulatory pathways for CD8 T lymphocyte activation. J Immunol 163, 1133-1142 (1999).
Sica, A., et al. Autocrine production of IL-10 mediates defective IL-12 production and NF-kappa B activation in tumor-associated macrophages. J Immunol 164, 762-767 (2000).
Siegal, F. P., et al. The nature of the principal type 1 interferon-producing cells in human blood. Science 284, 1835-1837 (1999).
Sikora, A. G., et al. IFN-alpha enhances peptide vaccine-induced CD8+ T cell numbers, effector function, and antitumor activity. J Immunol 182, 7398-7407 (2009).

Sinha, P, Clements, V K, Bunt, S K, Albelda, S M and Ostrand-Rosenberg, S (2007). Cross-talk between myeloid-derived suppressor cells and macrophages subverts tumor immunity toward a type 2 response. J Immunol 179: 977-983.
Soiffer, R., et al. Vaccination with irradiated autologous melanoma cells engineered to secrete human granulocyte-macrophage colony-stimulating factor generates potent antitumor immunity in patients with metastatic melanoma. Proc Natl Aced Sci USA 95, 13141-13146 (1998).
Sondergaard, H, Frederiksen, K S, Thygesen, P, Galsgaard, E D, Skak, K, Kristjansen, P E et al. (2007). Interleukin 21 therapy increases the density of tumor infiltrating CD8+ T cells and inhibits the growth of syngeneic tumors. Cancer Immunol Immunother 56: 1417-1428.
Stagg, J, Wu, J H, Bouganim, N and Galipeau, J (2004a). Granulocyte-macrophage colony-stimulating factor and interleukin-2 fusion cDNA for cancer gene immunotherapy. Cancer Res 64: 8795-8799.
Stagg, J, Lejeune, L, Paquin, A and Galipeau, J (2004). Marrow stromal cells for interleukin-2 delivery in cancer immunotherapy. Hum Gene Ther 15: 597-608.
Sukhanov, S, Higashi, Y, Shai, S Y, Vaughn, C, Mohler, J, Li, Y et al. (2007). IGF-1 reduces inflammatory responses, suppresses oxidative stress, and decreases atherosclerosis progression in ApoE-deficient mice. Arterioscler Thromb Vasc Biol 27: 2684-2690.

Thurner, B., et al. Vaccination with mage-3A1 peptide-pulsed mature, monocyte-derived dendritic cells expands specific cytotoxic T cells and induces regression of some metastases in advanced stage IV melanoma. J Exp Med 190, 1669-1678 (1999).
Viguier, M., et al. Foxp3 expressing CD4+CD25(high) regulatory T cells are overrepresented in human metastatic melanoma lymph nodes and inhibit the function of infiltrating T cells. J Immunol 173, 1444-1453 (2004).
Williams, P., Rafei, M., Bouchentouf, M., Raven, J., Yuan, S., Cuerquis, J., Former, K., Birman, E., and Galipeau, J. A Fusion of GMCSF and IL-21 Initiates Hypersignaling Through the IL-21Rα Chain With Immune Activating and Tumoricidal Effects In Vivo. Molecular Therapy (2010).
Weninger, W., Crowley, M. A., Manjunath, N. & von Andrian, U. H. Migratory properties of naive, effector, and memory CD8(+) T cells. J Exp Med 194, 953-966 (2001).

Wynes, M W and Riches, D W (2003). Induction of macrophage insulin-like growth factor-I expression by the Th2 cytokines IL-4 and IL-13. J Immunol 171: 3550-3559.

Yamamoto, M, Yoshizaki, K, Kishimoto, T and Ito, H (2000). IL-6 is required for the development of Th1 cell-mediated murine colitis. J Immunol 164: 4878-4882.

1. A conjugate protein comprising GM-CSF or a fragment thereof linked to IL-21 or a fragment thereof.

2. The conjugate protein according to claim 1 wherein the GM-CSF is linked to the IL-21 by a peptide linker, wherein the linker has 1 to 15 amino acids.

3. (canceled)

4. The conjugate protein according to claim 1, wherein the GM-CSF lacks the last 10 carboxy terminal amino acids.

5. The conjugate protein according to claim 4 which has the sequence shown in SEQ ID NO:2 or 4 or a homolog or analog thereof.

6. A nucleic acid molecule comprising a nucleic acid sequence encoding the conjugate protein of claim 1.

7. The nucleic acid molecule of claim 6 having the sequence shown in SEQ ID NO:1 or 3 or a homolog or analog thereof.

8. (canceled)

9. (canceled)

10. A method of treating cancer comprising administering an effective amount of the conjugate protein according to claim 1 or a nucleic acid encoding the conjugate protein to an animal or cell in need thereof.

11. The method of claim 10, wherein the cancer comprises IL-21R-expressing cells.

12. The method of claim 10, wherein the cancer comprises a non-hematological cancer.

13. The method of claim 12, wherein the non-hematological cancer is breast cancer or melanoma.

14. A method of enhancing or promoting cell death comprising administering an effective amount of the conjugate protein according to claim 1 or a nucleic acid encoding the conjugate protein to an animal or cell in need thereof.

15. A method of activating the immune response in an animal or cell in need thereof comprising administering an effective amount of the conjugate protein according to claim 1 or a nucleic acid encoding the conjugate protein.

16. A method of activating an immune response comprising administering ex vivo-treated monocytes, dendritic cells or macrophages to an animal in need thereof, wherein the monocytes, dendritic cells or macrophages have been treated ex vivo with the conjugate protein according to claim 1.

17. (canceled)

18. The method according to claim 10, wherein the GM-CSF lacks the last 10 carboxy terminal amino acids.

19. (canceled)

20. The method according to claim 10 wherein the GM-CSF or fragment thereof is linked to the IL-21 or fragment thereof by a peptide linker, wherein the linker has 1 to 15 amino acids.

21. (canceled)

22. The method according to claim 20 wherein the conjugate protein has the sequence shown in SEQ ID NO:2 or 4 or a homolog or analog thereof.

23. The method according to claim 10, wherein the animal is a human.

24. A pharmaceutical composition comprising an effective amount of the conjugate protein according to claim 1 or a nucleic acid molecule encoding the conjugate protein in admixture with a suitable diluent or carrier.

25. A pharmaceutical composition comprising an effective amount of ex vivo-treated monocytes, dendritic cells or macrophages in admixture with a suitable diluent or carrier, wherein the monocytes, dendritic cells or macrophages have been treated ex-vivo with the conjugate protein according to claim 1.

26. (canceled)

27. A screening assay for determining whether or not a compound is a tumoricidal agent comprising a) incubating the compound with cells that express IL-21R in the presence of the conjugate protein according to claim 1; and b) determining whether the compound competes with the conjugate protein; wherein competition with the conjugate protein indicates that the compound is a tumoricidal agent.

28. (canceled)


SEQUENCE LISTING (5 sequences)

SEQ ID NO: 1 - DNA, 846 bp, Artificial Sequence
  Synthetic construct
atgtggctgcagaatttacttttcctgggcattgtggtctacagcctctcagcacccacccgctcacccatcactgtcacccggccttggaagcatgtagaggccatcaaagaagccctgaacctcctggatgacatgcctgtcacgttgaatgaagaggtagaagtcgtctctaacgagttctccttcaagaagctaacatgtgtgcagacccgcctgaagatattcgagcagggtctacggggcaatttcaccaaactcaagggcgccttgaacatgacagccagctactaccagacatactgccccccaactccggaaacggactgtgaaacacaagttaccacctatgcggatttcatagacagccttaaaacctttctgactgatatcaccatggagaggacccttgtctgtctggtagtcatcttcttggggacagtggcccataaatcaagcccccaagggccagatcgcctcctgattagacttcgtcaccttattgacattgttgaacagctgaaaatctatgaaaatgacttggatcctgaacttctatcagctccacaagatgtaaaggggcactgtgagcatgcagcttttgcctgttttcagaaggccaaactcaagccatcaaaccctggaaacaataagacattcatcattgacctcgtggcccagctcaggaggaggctgcctgccaggaggggaggaaagaaacagaagcacatagctaaatgcccttcctgtgattcgtatgagaaaaggacacccaaagaattcctagaaagactaaaatggctccttcaaaagatgattcatcagcatctctcctagaacacatag

SEQ ID NO: 2 - protein, 278 aa, Artificial Sequence
  Synthetic construct
MWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDITMERTLVCLVVIFLGTVAHKSSPQGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLS

SEQ ID NO: 3 - DNA, 961 bp, Artificial Sequence
  Synthetic construct
atgactgccatgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagttcatgagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattcctgaggatctaacttgcagttggacagctagc

SEQ ID NO: 4 - protein, 305 aa, Artificial Sequence
  Synthetic construct
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEFMRSSPGNMERIVICLMVIFLGTLVHKSSSQGQDRHIRMRQIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 5 - protein, 9 aa, Rattus rattus
TYVPANASL